(19) United States
(12) Patent Application Publication
(10) Pub. No.: US 2025/0025189 A1
(43) Pub. Date: Jan. 23, 2025

(54) ASPIRATION SYSTEMS AND METHODS FOR REMOVAL OF UNDESIRED MATERIAL FROM A CIRCULATORY SYSTEM

(71) Applicant: ANGIODYNAMICS, INC., Latham, NY (US)

(72) Inventors: Kevin SWIFT, Hudson, MA (US); Mark GIRARD, Waltham, MA (US); Kenneth LEBLANC, Pepperell, MA (US)

(21) Appl. No.: 18/776,324

(22) Filed: Jul. 18, 2024

Related U.S. Application Data
(60) Provisional application No. 63/553,281, filed on Feb. 14, 2024.
(60) Provisional application No. 63/527,936, filed on Jul. 20, 2023.

(51) Int. Cl. A61B 17/22; A61M 1/00; A61M 1/36

(52) U.S. Cl. CPC A61B 17/22; A61M 1/3659; A61M 1/80; A61B 2017/22079

(57) ABSTRACT

Aspiration systems provided include first and second sheath assemblies, and a cannula. The first assembly includes a first shaft and hub. The first hub includes a hemostasis valve including a first multi-durometer sealing gland and a side port to be operatively coupled to a first suction source to generate a suction force. The cannula is configured to insert within the first assembly and be operatively coupled to a second suction source to generate another suction force. The first sealing gland is configured to selectively seal around the cannula or completely close when the cannula is not inserted. The second assembly includes a second shaft having a tapered distal end and second hub. The second hub includes a hemostasis valve having a second multi-durometer sealing gland. The first shaft is configured to insert into the second shaft, and the second sealing gland is configured to close around the first shaft.

### CROSS REFERENCE TO RELATED APPLICATIONS

This application claims the benefit of priority to U.S. Provisional Application No. 63/553,281 entitled “Improved Manual Mechanical Aspiration Device and Method of Use,” filed Feb. 14, 2024, as well as the benefit of priority to U.S. Provisional Application No. 63/527,936 entitled “Improved Manual Mechanical Aspiration Device and Method of Use,” filed Jul. 20, 2023, both of which are hereby incorporated herein by reference in their entirety.

This application is also related to commonly assigned U.S. patent application Ser. No. 17/170,782, filed Feb. 8, 2021, and issued as U.S. Pat. No. 11,648,020 on May 16, 2023, and U.S. patent application Ser. No. 18/309,223, filed Apr. 28, 2023, and published Oct. 19, 2023, as Publication No. 2023/0329730, as well as commonly assigned U.S. patent application Ser. No. 18/503,864 filed Nov. 7, 2023, and published May 9, 2024, as Publication No. 2024/0148956, entitled “Filter For Mechanical Thrombectomy Device And Method of Using the Same,” commonly assigned U.S. patent application Ser. No. 16/778,657, filed Jan. 31, 2020, and published May 28, 2020 as Publication No. 2020/0164117, and commonly assigned U.S. patent application Ser. No. 17/869,687, filed Jul. 20, 2022, and published Feb. 2, 2023, as Publication No. 2023/0030606, each of which is hereby incorporated herein by reference in its entirety.

### BACKGROUND

This disclosure relates generally to aspiration systems and methods for removing undesired material from a site of interest within a circulatory system, and more particularly, to enhanced mechanical aspiration systems and methods.

Patients can suffer from the presence of undesired material, most notably blood clots, in the circulatory system, including, but not limited to, in blood vessels and heart chambers. Examples of such disease include, but are not limited to, myocardial infraction, stroke, pulmonary embolism, deep venous thrombosis, atrial fibrillation, infective endocarditis, etc. The undesired intravascular material can include blood clots, tumors, infected vegetations, foreign bodies, etc. Blood clots can arise spontaneously within a blood vessel or heart chamber (thrombosis) or be carried through the circulatory system from a remote site and lodge in a blood vessel (thromboemboli).

There are a variety of techniques for removing undesired material from the circulatory system including, for instance, the delivery of pharmaceutical agents (such as thrombolytic agents), mechanical treatments (such as aspiration and/or mechanical maceration), catheter-based removal techniques (such as catheter pulmonary embolectomy), as well as other general surgical treatments.

### SUMMARY

Certain shortcomings of the prior art are overcome, and additional advantages are provided herein through the provision of an aspiration system which includes a first sheath assembly, a cannula and a second sheath assembly. The first sheath assembly includes a first elongate shaft and a first hub at a proximal end of the first elongate shaft. The first hub includes a hemostasis valve including a first multi-durometer sealing gland and a side port configured to be operatively coupled to a first suction source to generate a suction force through the first elongate shaft. The cannula includes a cannula shaft configured to be inserted within the first sheath assembly and to be operatively coupled to a second suction source to generate a suction force through the cannula. The first multi-durometer sealing gland is configured to selectively close to seal around the cannula shaft or completely close when the cannula is not positioned within the first sheath assembly. The second sheath assembly includes a second elongate shaft having a tapered distal end and a second hub at a proximal end of the second elongate shaft. The second hub includes another hemostasis valve having a second multi-durometer sealing gland. The first elongate shaft of the first sheath assembly is configured to be inserted into the second elongate shaft of the second sheath assembly and the second multi-durometer sealing gland of the second sheath assembly is configured to seal around the first elongate shaft of the first sheath assembly.

In another aspect, a method for removing an undesired material from a vasculature is provided. The method includes providing an aspiration system with a distal end proximate the undesired material. The aspiration system includes a first sheath assembly, a cannula and a second sheath assembly. The first sheath assembly includes a first elongate shaft and a first hub at a proximal end of the first elongate shaft. The first hub includes a hemostasis valve including a first multi-durometer sealing gland and a side port configurated to be operatively coupled to a first suction source. The cannula includes a cannula shaft configured to be inserted within the first sheath assembly and to be operatively coupled to a second suction source. The first multi-durometer sealing gland is configured to selectively close to seal around the cannula shaft or completely close when the cannula is not positioned within the first sheath assembly. The second sheath assembly includes a second elongate shaft having a tapered distal end and a second hub at a proximal end of the second elongate shaft. The second hub includes another hemostasis valve having a second multi-durometer sealing gland. The first elongate shaft of the first sheath assembly is configured to be inserted into the second elongate shaft of the second sheath assembly and the second multi-durometer sealing gland of the second sheath assembly is configured to seal around the first elongate shaft of the first sheath assembly. In addition, the method includes actuating the second suction source to generate a suction force through the cannula shaft to remove a first portion of the undesired material from a vessel of the vasculature, and removing the cannula shaft from the first sheath assembly. Further, the method includes actuating the first suction source to generate a suction force through the first elongate shaft to remove a second portion of the undesired material from the vessel of the vasculature via the side port of the first hub of the first sheath assembly.

Additional features and advantages are realized through the techniques described herein. Other embodiments and aspects are described in detail herein and are considered a part of the claimed aspects.

### BRIEF DESCRIPTION OF THE DRAWINGS

One or more aspects are particularly pointed out and distinctly claimed as examples in the claims at the conclusion of the specification. The foregoing and objects, features, and advantages of one or more aspects are apparent from the following detailed description taken in conjunction with the accompanying drawings in which:

FIGS. 1A-1C illustrate one embodiment of an aspiration system, in accordance with one or more aspects of the present disclosure;

FIG. 2 illustrates a partial cross-sectional embodiment of the cannula of the aspiration system of FIG. 1A-1C, and showing the cannula connection hub with side port (or Y-port), in accordance with one or more aspects of the present disclosure;

FIGS. 3A-3C illustrate in greater detail one embodiment of the introducer sheath assembly of the aspiration system of FIGS. 1A-1B, in accordance with one or more aspects of the present disclosure;

FIGS. 4A-4B illustrate further details of one embodiment of the connection hubs coupling the cannula, the cannula sheath assembly, and the introducer sheath assembly of the aspiration system of FIGS. 1A-1B, in accordance with one or more aspects of the present disclosure;

FIG. 4C illustrates another embodiment of the connection hubs coupling a connector, the cannula sheath assembly, and the introducer sheath assembly, where the connector is configured to operatively couple to an aspiration source, in accordance with one or more aspects of the present disclosure;

FIG. 5 illustrates one embodiment of a multi-durometer sealing gland of a hemostasis valve of a connection hub, such as the cannula sheath hub and/or introducer sheath hub of FIGS. 4A-4B, in accordance with one or more aspects of the present disclosure;

FIGS. 6A-6C illustrate another embodiment of an aspiration system, in accordance with one or more aspects of the present disclosure;

FIGS. 7A-7B depict partial alternate embodiments of aspiration systems for aspirating via a cannula sheath side port of a cannula sheath hub such as depicted in FIGS. 6A-6C, in accordance with one or more aspects of the present disclosure;

FIGS. 8A-8C illustrate further embodiments of an aspiration system, in accordance with one or more aspects of the present disclosure;

FIGS. 9A-9B illustrate a collapsible lumen, in open and closed positions, for a cannula shaft or a cannula sheath shaft of an aspiration system, in accordance with one or more aspects of the present disclosure;

FIG. 10 depicts one embodiment of a method of aspiration via a cannula sheath side port of a cannula sheath hub of an aspiration system, such as depicted in FIGS. 8A-8B, in accordance with one or more aspects of the present disclosure;

FIG. 11 depicts another embodiment of a method of aspiration via a cannula sheath side port of a cannula sheath hub of an aspiration system, such as depicted in FIGS. 8A-8B, in accordance with one or more aspects of the present disclosure;

FIG. 12 depicts a further embodiment of a method of aspiration via a cannula sheath side port of a cannula sheath hub of an aspiration system, such as depicted in FIGS. 8A-8B, in accordance with one or more aspects of the present disclosure;

FIG. 13 depicts still another embodiment of a method of aspiration via a cannula sheath side port of a cannula sheath hub of an aspiration system, such as depicted in FIGS. 8A-8B, in accordance with one or more aspects of the present disclosure;

FIG. 14 depicts one embodiment of a method of injecting contrast/flush/filtered blood via a collapsible lumen of a cannula or cannula sheath shaft of an aspiration system, such as depicted in FIGS. 1A-1B & 6A-9B, in accordance with one or more aspects of the present disclosure;

FIG. 15 depicts one embodiment of a method of reinfusion of blood through an introducer sheath of an assembled aspiration system, such as depicted in FIGS. 1A-1B & 6A-8B, in accordance with one or more aspects of the present disclosure;

FIG. 16 depicts one embodiment of a method for removing, at least in part, an undesired material from a vasculature and reinfusing blood through an introducer sheath of an assembled aspiration system, such as depicted in FIGS. 1A-1C, in accordance with one or more aspects of the present disclosure;

FIG. 17 depicts one embodiment of a method of removing, at least in part, an undesired material from a vasculature and reinfusing blood through an introducer sheath of an assembled aspiration system, such as depicted in FIGS. 6A-6B, in accordance with one or more aspects of the present disclosure;

FIG. 18 depicts one embodiment of a method of removing, at least in part, an undesired material from a vasculature and reinfusing blood through an introducer sheath of an assembled aspiration system, such as depicted in FIGS. 8A-8B, in accordance with one or more aspects of the present disclosure;

FIGS. 19A-19B illustrate embodiments of the aspiration system of FIGS. 1A-1B being deploying within a patient for removing an undesired material from a treatment site, in accordance with one or more aspects of the present disclosure; and

FIG. 20 depicts one embodiment of a method of aspirating an undesired material from a treatment site using a deployed aspiration system, such as depicted in FIGS. 19A-19B, in accordance with one or more aspects of the present disclosure.

### DETAILED DESCRIPTION

Aspects of the present disclosure and certain features, advantages, and details thereof, are explained more fully below with reference to the non-limiting examples illustrated in the accompanying drawings. Descriptions of well-known systems, devices, materials, fabrication techniques, aspiration methods, etc., are omitted so as not to unnecessarily obscure the disclosure in detail. It should be understood, however, that the detailed description and the specific examples, while indicating aspects of the disclosure, are given by way of illustration only, and are not by way of limitation. Various substitutions, modifications, additions, and/or arrangements, within the spirit and/or scope of the underlying inventive concepts will be apparent to those skilled in the art for this disclosure. Note further that reference is made below to the drawings, where the same or similar reference numbers used throughout different figures designate the same or similar components. Also, note that numerous inventive aspects and features are disclosed herein, and unless otherwise inconsistent, each disclosed aspect or feature is combinable with any other disclosed aspect or feature as desired for a particular application of the concepts disclosed.

As used herein, the term “about” means plus or minus 10% of the numerical value of the number with which it is being used. Therefore, about 50 mm means in the range of 45 mm to 55 mm. Also, the terms “patient” and “subject” include, but are not limited to, humans and animals.

Note also that “undesired intravascular material” or “undesired material” refers to any intravascular debris including, but not limited to, thrombus; embolus; clot; vegetative growth; infected vegetative growth (such as endocarditis); pulmonary embolism; tumor; arterial clots, undesired material trapped in dialysis grafts and/or stents, and other undesired natural and/or unnatural foreign bodies to be removed from a subject's body.

The phrases “manual mechanical aspiration source”, “manually-operated aspiration device”, “aspiration device”, and “thrombectomy device” are used interchangeably herein to refer to any of variety of manually-operated devices used to facilitate removal of undesired material from a vasculature, such as via aspiration. Various examples of such devices are described in detail in the above-incorporated, commonly assigned U.S. Pat. No. 11,648,020.

En bloc refers to entirely, wholly, and/or without significant fragmentation.

A suction force and/or vacuum force refers to a negative pressure created by removing air from a space creating a pressure differential resulting in the force that a vacuum exerts upon the undesired intravascular material. A drive force refers to the pressure differential generated by the device that exerts a force, such as upon the undesired intravascular material.

Differential pressure is the difference in pressure between two given points. Positive pressure refers to the pressure at a first point that is greater than pressure at a second point. Negative pressure refers to a pressure at a first point that is lower than pressure at a second point.

A vacuum as used herein refers to a differential pressure, including decreases in pressure (negative pressure) below atmospheric pressure and increases in pressure (positive pressure) above atmospheric bidirectional differential pressure. By way of specific example only, a vacuum or negative pressure for the suction force can range from −11 psi to −14.7 psi, and a positive pressure for the driving force can range from +1 psi to +10 psi (i.e., the range of the return spring force).

A trigger pull cycle refers to the combined retraction or compression and release of the trigger assembly of a manual mechanical aspiration source. Fully retracted is defined as a maximum distance of travel for the trigger assembly starting from a rest position and/or deactivated state. Partially retracted is defined as any distance between the trigger assembly at a rest position and/or deactivated state and a full retraction of the trigger assembly, i.e., some distance less than the possible maximum distance of travel for the trigger assembly starting from a rest position and/or deactivated state.

Target vessels, treatment sites, or target areas include, but are not limited to, systemic venous circulation (e.g., inferior vena cava and/or superior vena cava, pelvic veins, leg veins, neck and arm veins); arterial circulation (e.g., aorta or its large and medium branches); heart chambers, such as in the left heart (e.g., the left ventricular apex and left atrial appendage), in the right heart (e.g., right atrium and right ventricle), or on its valves; small blood vessels; medium blood vessels; large blood vessels; iliofemoral vein; peripheral vasculature; and/or the pulmonary circulation (e.g., pulmonary veins and/or pulmonary arteries). In some embodiments, other treatment sites or target areas could include other nonvascular tubular structures, such as ducts or any other avascular tubular tissue. In some embodiments, other treatment sites or target areas could include, pacemaker leads, stents, or other artificial implanted medical devices.

This disclosure relates to enhanced aspiration systems and methods for minimally invasive removal of undesired material from a vessel or other hollow anatomical structure of a subject. More specifically, in one or more embodiments, this disclosure relates to aspiration systems and methods which include, or use, a first sheath assembly, a cannula, and a second sheath assembly, as well to aspiration systems and methods which include, or use, only a first sheath assembly and second sheath assembly, where aspiration and removal of the undesired material occurs, at least in part, through the first sheath assembly. Further, in one or more embodiments, this disclosure relates to aspiration systems which facilitate removal of an undesired material using, for instance, one or more disposable, manually-operated aspiration devices (or manual mechanical aspiration sources) coupled to, and in fluid communication with, a cannula and/or a cannula sheath assembly, as well as to, for instance, a waste assembly and/or filter assembly. The cannula and/or cannula sheath assembly can include an expandable funnel at a distal end to aid in the removal of the undesired intravascular material. The manually-operated aspiration device(s) provide for single-handed operation and manual control of generating a suction force and/or a drive force within the cannula and/or cannula sheath shaft during the removal of the undesired material from a subject.

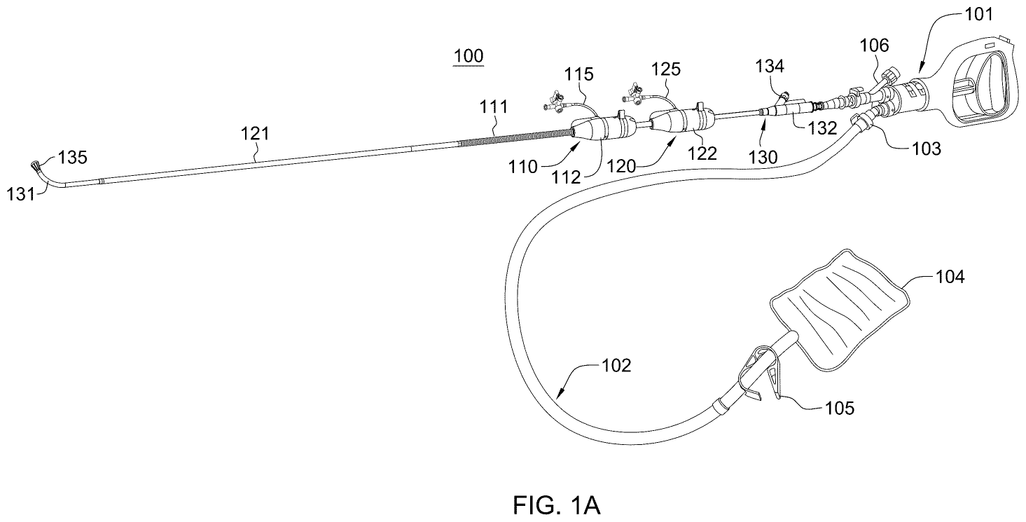

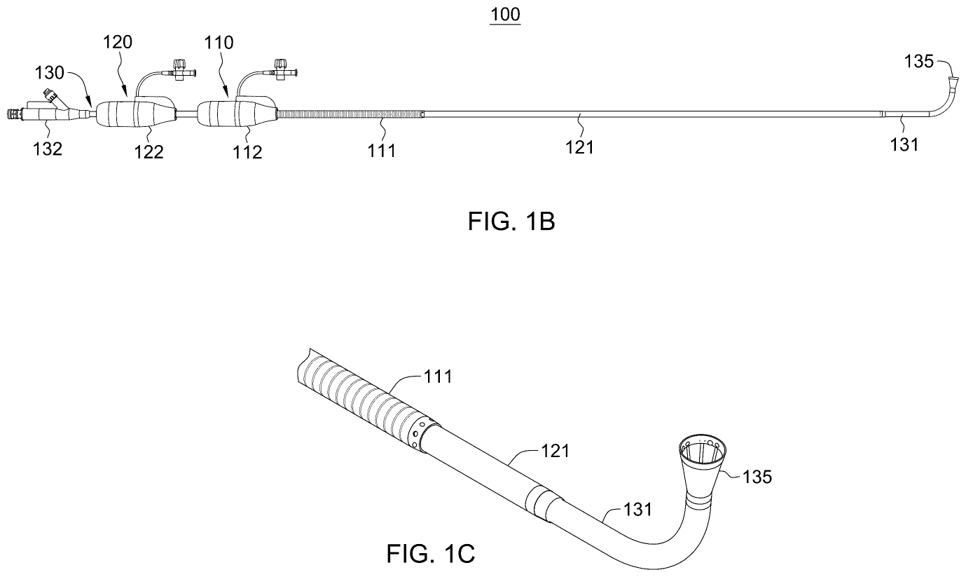

Aspiration systems such as disclosed herein are designed to facilitate removal of undesired intravascular material from a subject. Multiple enhanced aspiration systems, and aspiration system aspects, are discussed herein with reference to FIGS. 1A-9B, by way of example only. In one or more embodiments, the aspiration systems disclosed are enhanced manual mechanical aspiration systems configured to facilitate removal of undesired intravascular material using one or more manually controlled handheld devices configured to aspirate and/or remove blood, fluid, or undesired material from the circulatory system of a subject. In one or more embodiments, the undesired intravascular material is filtered from the removed fluid, such as described in the above-incorporated, commonly assigned US Patent Publication No. 2024/0148956. As noted, further details of one or more mechanical thrombectomy devices usable with the enhanced aspiration systems described herein are described in the above-incorporated US Patent Publication Nos. 2023/0329730 and 2024/0148956.

Referring collectively to FIGS. 1A-4B, various aspects of an aspiration system 100 are disclosed, which include, or couple to, one or more aspiration devices 101 (FIG. 1A) or aspiration sources. In one or more embodiments, aspiration system 100 can be configured, or used, to remove an undesired intravascular material either en bloc or in stages using, for instance, one or more aspiration devices or sources (such as syringes and/or manual or non-manual pumps, including roller pumps, peristatic pumps, centrifugal pumps, wall pumps, etc.) coupled to, and in fluid communication with, a cannula and waste assembly, and/or coupled to, and in fluid communication with a cannula sheath assembly of the system, such as described herein.

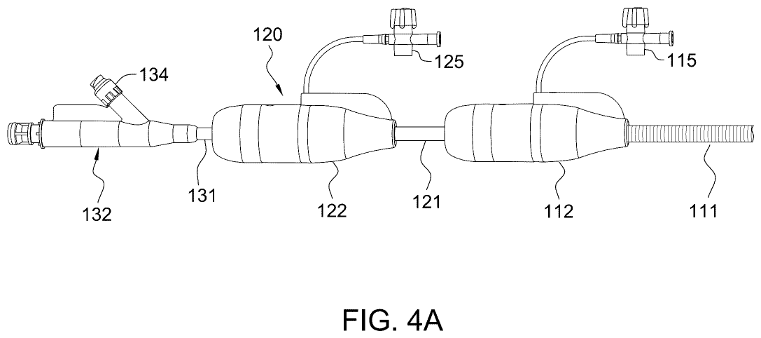

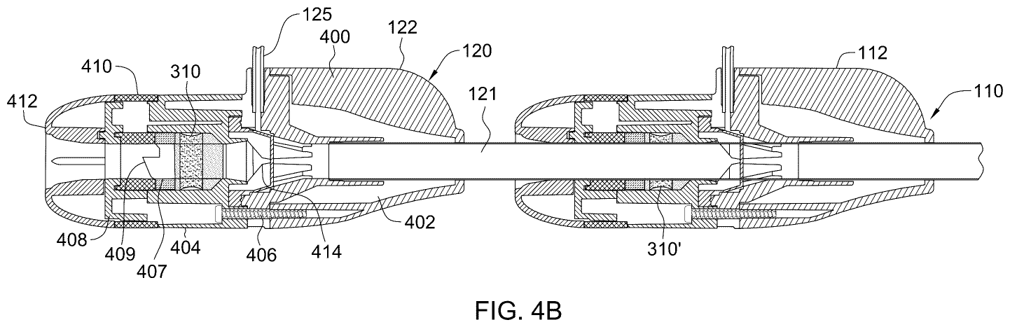

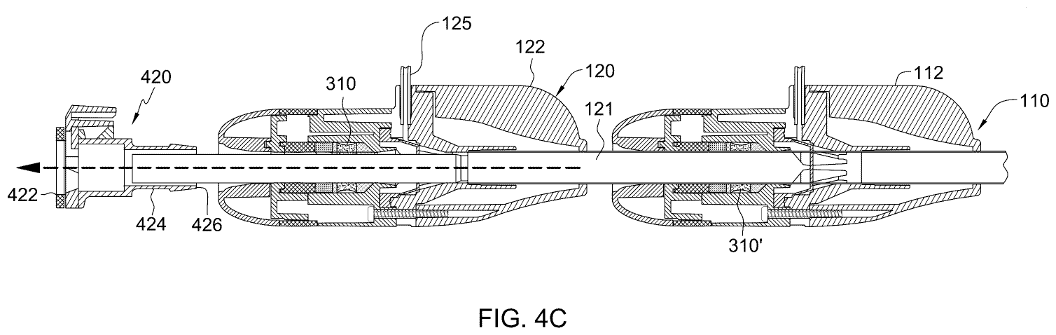

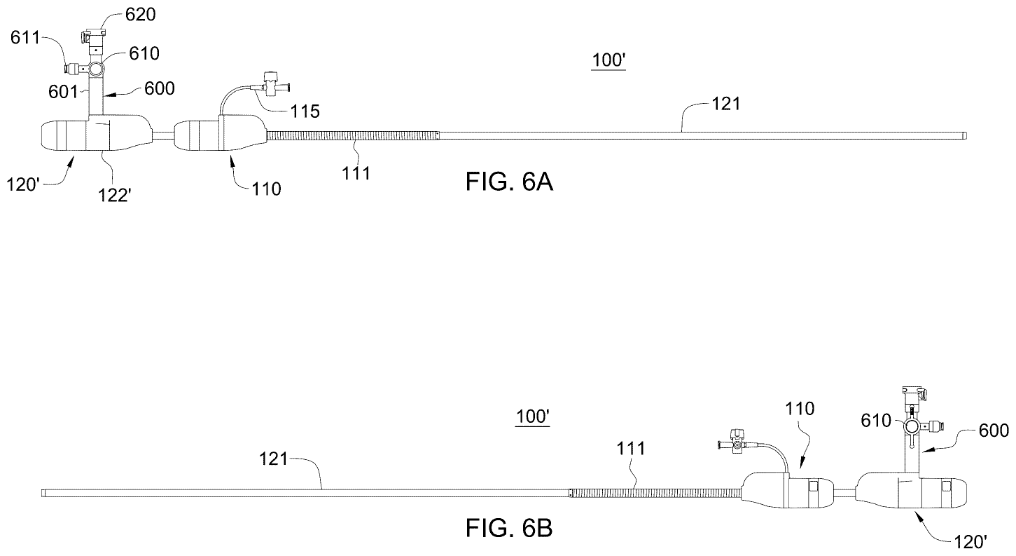

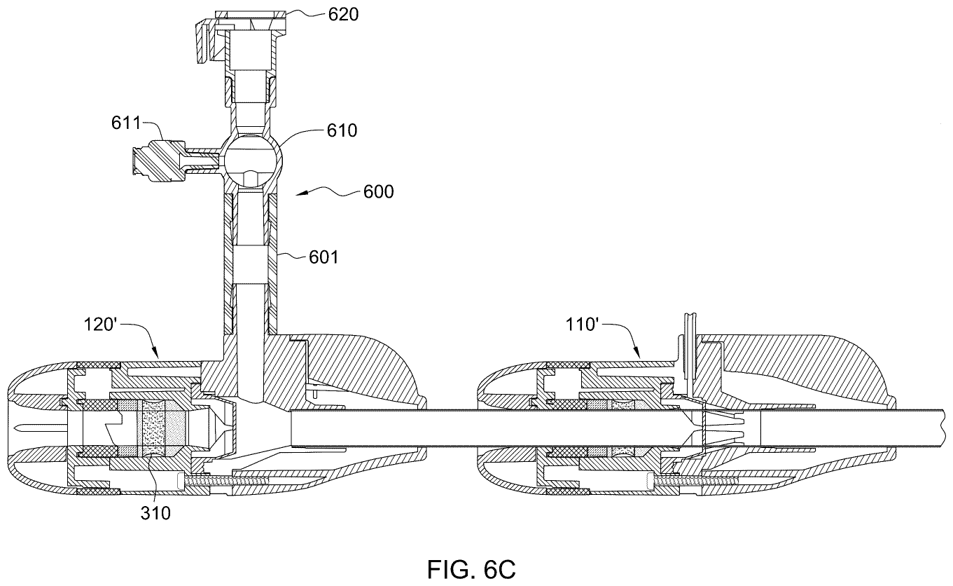

By way of example, in the depicted embodiment, aspiration system 100 includes a cannula sheath assembly 120 (or first sheath assembly), including a cannula sheath 121 (or first elongate shaft), and a cannula sheath connection hub 122 (or first hub), at a proximal end of cannula sheath 121. Cannula sheath 121 includes at least one lumen or central opening. In one or more embodiments, cannula sheath connection hub 122 includes a hemostasis valve including a first multi-durometer sealing gland 310 (or membrane, valve, or seal) (FIG. 4B) and a side port 125 (or side arm assembly) configured to operatively inject a fluid through cannula sheath 121. The side port 125 can have a bore size of approximately 4 F to 5 F. However, it is to be understood, that the side port 125 can have any bore size capable of allowing the injection of fluid as desired by the clinician or needed for the treatment. In other embodiments, as will be discussed herein, a large-bore side port 600 can be used in place of or in addition to side port 125 and is configured to be coupled to a suction source to generate a suction force through, for instance, the cannula sheath 121 (see FIGS. 6A-8B).

In one or more embodiments, cannula 130 of aspiration system 100 includes a cannula shaft 131 with at least one lumen or elongate opening. Cannula shaft 131 is configured to be inserted within cannula sheath assembly 120 and be operatively coupled via a proximal end connection hub 132 to a suction source, such as a manual mechanical aspiration device, such as manual mechanical aspiration source 101 of FIG. 1A, to generate a suction force through the cannula shaft 131. Note that the suction force can be generated by a variety of mechanical devices including, for instance, a syringe coupled in fluid communication with a cannula 130 of aspiration system 101 in FIG. 1A. However, in other embodiments, the suction force can be generated by any of the aspiration devices or sources discussed elsewhere herein.

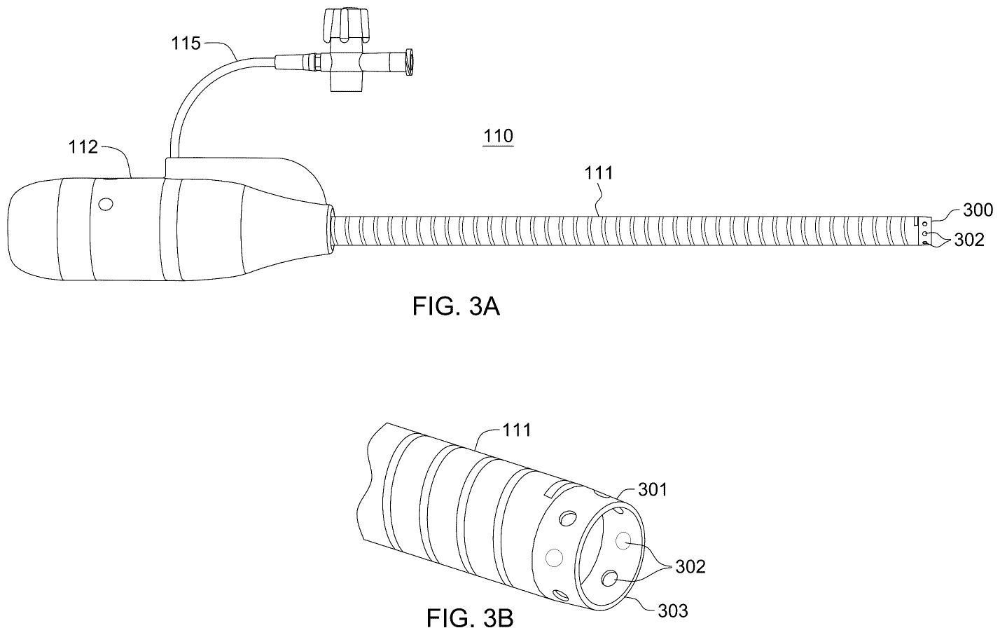

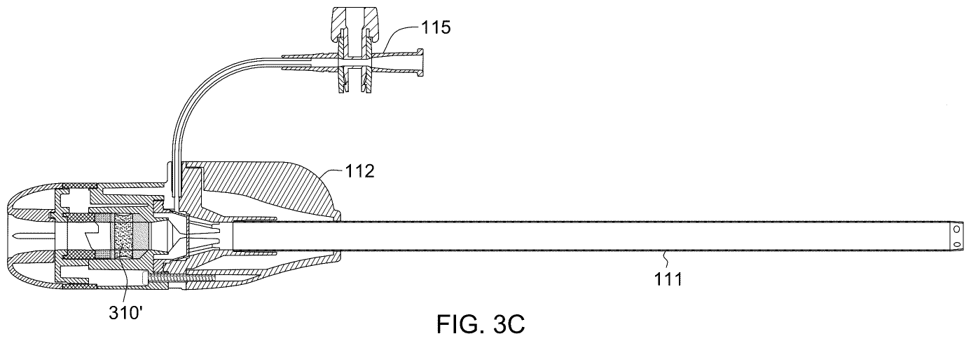

As illustrated, aspiration system 100 further includes, in one or more embodiments, an introducer sheath assembly 110 (or second sheath assembly), which includes an introducer sheath 111 (or second elongate shaft), having at least one lumen or elongate opening 300 (FIG. 3B), and a tapered distal end 301 (FIG. 3B). The tapered distal end 301 may transition the lumen of the introducer sheath 111 to approximately the size of the outer diameter of the cannula sheath 121 such that the inner diameter of the lumen of the introducer sheath 111 is larger than the inner diameter of the opening 300 at the distal most end of the tapered distal end 301 of the introducer sheath 111. In this way, the tapered distal end 301 can seal around the cannula sheath 121 in some embodiments. In other embodiments, there can be a space or gap between the distal most end of the tapered distal end 301 and the outer surface of the introducer sheath 111 to further allow for injection or reinfusion. In addition, introducer sheath assembly 110 includes an introducer sheath connection hub 112 (or second hub), at a proximal end of the introducer sheath 111. In one or more embodiments, introducer sheath connection hub 112 is similar to, or even the same as (in certain embodiments), cannula sheath connection hub 122 of cannula sheath assembly 120. In one or more embodiments, the introducer sheath connection hub 112 includes a hemostasis valve having a second multi-durometer sealing gland 310′ (FIG. 3C), which in one embodiment, can be similar to multi-durometer sealing gland 310 of cannula sheath connection hub 122 of cannula sheath assembly 120.

Note that the first multi-durometer sealing gland 310 (FIG. 4B) of the hemostasis valve of the cannula sheath connection hub 122 is configured to selectively close to seal around cannula shaft 131 when assembled such as depicted in FIGS. 1A-1C, or another device inserted in its place, such as a guidewire, or to completely close when cannula 130 (or another device) is not positioned within cannula sheath assembly 120. Note also that the cannula sheath 121 of cannula sheath assembly 120 is configured and sized to be inserted into introducer sheath 111 of introducer sheath assembly 110, and the second multi-durometer sealing gland 310′ of introducer sheath assembly 110 is configured to seal around cannula sheath 121 of cannula sheath assembly 120 when operatively assembled, such as depicted in FIGS. 1A-4B.

In one or more embodiments, one or more, or each, of cannula sheath assembly 120, cannula 130 and introducer sheath assembly 110 include one or more side ports, such as side access port 134 of connection hub 132 of cannula 130, as well as side port 125 of cannula sheath hub 122 and side port 115 of introducer sheath hub 112. In one embodiment, the one or more side ports can be one or more small-bore side ports, such as 4F-5F size. Alternatively, one or more other large-bore side ports, such as 16F-28F size, can be associated with one of the connection hubs in place of or in addition to the respective side ports. However, it is to be understood that the large-bore side port can have any bore size capable of allowing enough suction force to be generated within the lumen of the side port 600 to aspirate the undesired material. Example large-bore side ports are depicted in FIGS. 6A-8C, and described further below. In one or more embodiments, the large-bore side port configurations facilitate attaching a mechanical aspiration source, such as aspiration device/source 101 of FIG. 1A or other aspiration source, such as a syringe, to the side port of the respective connection hub. Note that in each instance, the side ports of the connection hubs of an aspiration system, in accordance with one or more aspects of the present disclosure, including any large-bore side port, are in fluid communication through the respective connection hubs with their respective lumens of the aspiration system, such as the cannula, cannula sheath, and introducer sheath depending on the connection hub.

As noted, aspiration system 100 includes, in one or more embodiments, introducer sheath assembly 110 (or second sheath assembly), which includes introducer sheath 111 (or second elongate shaft), having a lumen or elongate cavity 300 (FIG. 3B) extending there through and a tapered distal end 301 (FIG. 3B). In addition, introducer sheath assembly 110 includes introducer sheath connection hub 112 (or second hub), at a proximal end of introducer sheath 111. In one or more embodiments, introducer sheath connection hub 112 can be similar to, or the same as, cannula sheath connection hub 122 of cannula sheath assembly 120, or different depending on the aspiration system and methods to be used. Introducer sheath connection hub 112 also includes a hemostasis valve having second multi-durometer scaling gland 310′ (FIG. 3C), which, in one or more embodiments, can be similar to multi-durometer sealing gland 310 of the hemostasis valve of cannula sheath connection hub 122. The cannula sheath 121 of the cannula sheath assembly 120 is sized and configured to be inserted into and pass through connection hub 112 and introducer sheath 111 of introducer sheath assembly 110, and the second multi-durometer sealing gland 310′ of introducer sheath assembly 110 is configured to controllably seal around cannula sheath 121 of cannula sheath assembly 120 when operatively assembled, such as depicted in FIGS. 1A-4B, and described further below.

In one or more embodiments, introducer sheath assembly 110 includes an introducer sheath 111 that is a reinforced sheath having a lumen extending therethrough from a proximal end to a distal end. The sheath can include, for example, a coil, a braided tube or any other known or later developed tube reinforcing mechanism. Reinforcing the introducer sheath 111 enables for increased strength to insert and navigate the introducer sheath through the vasculature. At the proximal end, the introducer sheath hub 112 contains a hemostasis valve including a multi-durometer sealing gland, such as described herein. In some embodiments, the hemostasis valve can include silicone. The introducer sheath connection hub 112 can also contain a lever to control opening and closing of the multi-durometer sealing gland. As noted, the hub can have a side port 115 for fluid, such as flush, saline, contrast, thrombolic agents, medicine and/or blood, injection via, for example, a syringe. The distal end of the introducer sheath 111 is tapered to provide a gradual transition from the obturator to the introducer sheath. The tapered distal end also provides smooth transition to the cannula sheath of the aspiration system. The distal end of the introducer sheath 111 can include radiopaque markers to aid in visibility of the introducer sheath 111 via an imaging device. The tapered distal end 301 can include an unreinforced area including at least one side hole 302. In some embodiments, the at least one side hole 302 can include a plurality of side holes arranged around the distal end of the introducer sheath 111. Further, in some embodiments, the side hole(s) can include a slit valve, such as a pressure activated slit valve or opening. The side holes can be configured to facilitate injection of flush, saline, contrast, thrombolic agents, medicine and/or reinfusion of blood from the side port 115 of the introducer sheath connection hub 112. The tapered distal end 301 can transition the lumen of the introducer sheath 111 to approximately the size of the outer diameter of the cannula sheath 121 such that the inner diameter of the lumen of the introducer sheath 111 is larger than the inner diameter of the opening 300 at the distal most end of the tapered distal end 301 of the introducer sheath 111. In this way, the tapered distal end 301 seals around the cannula sheath 121. However, the inner diameter of the introducer sheath 111 along its length may be larger than the outer diameter of the cannula sheath 121 such that there is a space or gap between the inner surface of the introducer sheath 111 and the outer surface of the cannula sheath 121 in order to allow the fluid that is being injected into the introducer sheath 111 to flow therebetween. Additionally, the tapered distal end 301 may be configured such that there is a space or gap between the inner surface of the tapered distal end 301 at a position of the opening(s) 302 in order to ensure that fluid that is being injected into the introducer sheath 111 can flow through the openings 302. In this way, blood (such as filtered blood removed via aspiration with the undesired material that was removed using the aspiration system 100) can be reinfused directly into the vasculature from which it was removed while the cannula sheath assembly 120 is positioned within the introducer sheath assembly 110.

As noted, in one or more embodiments, one or more, or each, of cannula sheath assembly 120, cannula 130 and introducer sheath assembly 110 includes a connection hub with one or more side ports, or side access ports, such as side access port 134 of cannula 130, side port 125 of cannula sheath assembly 120 and side port 115 of introducer sheath assembly 110. In one or more embodiments, the side ports are configured as injection ports to facilitate injection of a fluid, such as a flush, saline, contrast, thrombolic agents, medicine, blood, and/or to introduce an accessory or ancillary device into the respective lumen. Additionally, in one or more other embodiments, one or more side ports can be associated with one or more of the connection hubs in place of or in addition to the respective injection port. Example side ports are depicted in FIGS. 6A-8B, and described further below. In one or more embodiments, the large-bore side port configurations facilitate attaching a manual mechanical aspiration device, such as manual mechanical aspiration source 101, or a syringe, to the respective connection hub through the side of the hub to, for instance, perform aspiration.

In some embodiments, the cannula shaft 131, cannula sheath 121 and/or introducer sheath 111 can be fabricated of Pebax or urethane. Further, in some embodiments, the cannula shaft 131, cannula sheath 121 and/or introducer sheath 111 can include a liner composed of, for example, PTFE or a hydrophilic material. Further, in some embodiments, the cannula shaft 131, cannula sheath 121 and/or introducer sheath 111 can include a reinforcement wire composed of, for example, a stainless steel or nitinol.

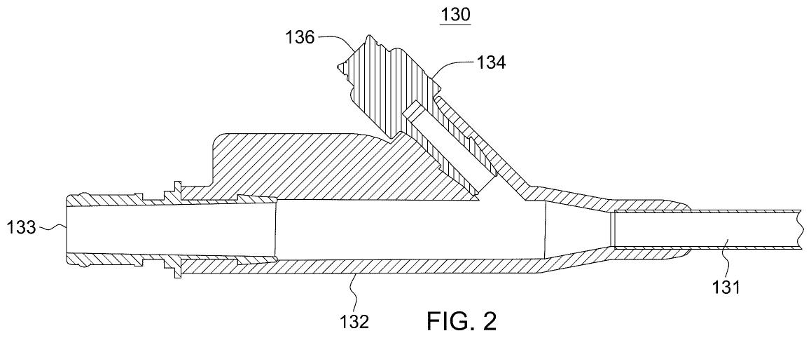

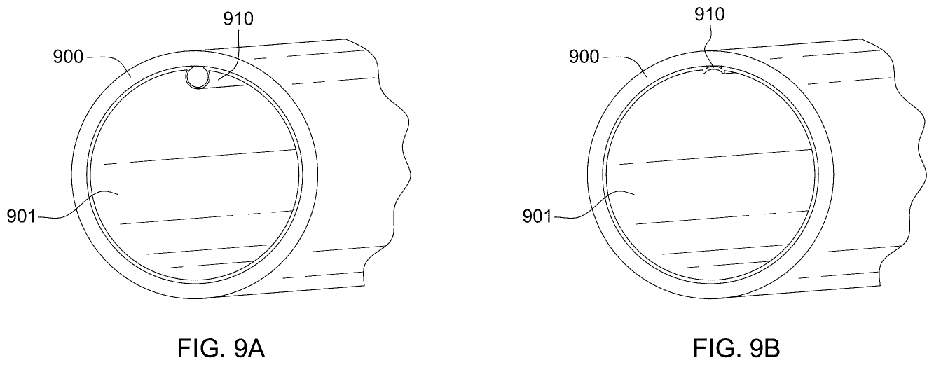

FIG. 2 is a cross-sectional elevational view of one embodiment of cannula 130. In this embodiment, cannula 130 includes cannula hub 132 at the proximal end of cannula 130. Cannula hub 132 includes, in one embodiment, a side port 134, such as a Y-port. In the depicted embodiment, cannula hub 132 thus includes one port, such as proximal port 133 at a proximal end of cannula hub 132, which is configured to connect to a manual mechanical aspiration source or aspiration device. In one embodiment, side port 134 further includes a port connector 136, in fluid communication with the cannula shaft. In one or more embodiments, port connector 136 can be configured to facilitate connection thereto for injection of a fluid, such as a flush or contrast, saline, thrombolic agents, medicine, blood and/or to introduce an accessory or ancillary device into the cannula shaft, such as a retriever device (such as described in the above-incorporated US Patent Publication No. 2023/0030606), a secondary treatment device, balloon catheter, angiographic catheter, embolic protection device, wire and the like, or another device, such as a transducer and/or a sensor for real time monitoring, such as pressure monitoring or heart rate monitoring. In one or more embodiments, the configuration of cannula 130 can be the same or different in various embodiments of an aspiration system, such as described herein. For instance, in one or more embodiments, the cannula shaft of cannula 130 can include or define one lumen, or can include or define multiple lumens. In one or more other embodiments, the cannula shaft can include a collapsible lumen such as described herein with reference to FIGS. 9A-9B. In other embodiments, different configurations of the cannula hub 132 can be utilized in an aspiration system in accordance with one or more aspects of the present disclosure.

As noted, in one or more embodiments, proximal port 133 of cannula 130 can be configured for attachment of a manual mechanical aspiration source, such as manual mechanical aspiration source 101 or a manual mechanical aspiration source disclosed in the above-incorporated U.S. Pat. No. 11,648,020. Briefly described, in one or more embodiments, the manually-operated aspiration device provides for single hand operation and manual control in generating a suction force and/or drive force during removal of an undesired intravascular material from a subject. Additionally, a waste assembly 102 can be coupled in fluid communication with the manually-operated aspiration device, as illustrated in FIG. 1A.

In one or more embodiments, the manually-operated aspiration source 101 includes a trigger assembly that has a resting position, as well as a partially-activated position and a fully-activated position. When in the resting position, no aspiration or suction force is generated by the aspiration device's pump assembly. When the trigger assembly is moved by a user to the partially-active position, the aspiration device is configured to generate an aspiration force or a suction force through the cannula 130 to aspirate at least a portion of undesired material. As the user releases grip on the trigger assembly, the trigger assembly is configured to move, as a result of a spring force generated by a spring within the device, from the partially-active position towards the resting position, and in so doing, to generate a drive force through connection tube 102 such that the undesired material that was aspirated is transferred through the connection tube 102 to the reservoir 104.

As illustrated in FIG. 1A, in one embodiment, manual mechanical aspiration source 101 can include a waste port 103 operatively coupled to a waste assembly, which in one embodiment, includes a connection tube 102 and a reservoir 104. In one or more embodiments, releasing the trigger of the aspiration device results in the pump assembly generating a drive force through the waste assembly port that removes aspirated undesired material from the pump assembly into the waste assembly. In one or more embodiments, aspiration device 101 includes a barrel outlet valve that remains closed until a certain cracking pressure or predetermined pressure threshold is reached. For instance, the predetermined cracking pressure can be high enough to withstand normal blood pressure or at least 30 mm of mercury. When pressure drops below the predetermined cracking pressure, the barrel outlet valve closes to prevent unwanted backflow or passive flowing of fluid into the waste collection assembly. When a driving force is generated, the barrel outlet valve opens to allow fluid flow from the barrel cavity of the manual mechanical aspiration source 101 through the waste port into the waste assembly. In one embodiment, the waste port connecter and cannula port connector of the aspiration source are differently dimensioned to prevent an operator inadvertently connecting the cannula to the waste port connector and/or waste collecting tube to the cannula port connector. In one or more embodiments, one or more pinch clamps 105 can be provided to temporarily block fluid flow through the waste collection assembly tubing, for instance, in the event that waste collection receptacle 104 becomes full or otherwise needs replacement. Once a new waste collection receptacle has been connected, the pinch clamp(s) can be opened to restore flow through the waste collection assembly tubing.

In one or more embodiments, a filter can be associated with the waste collection assembly, such as within a reservoir or waste container, such as disclosed in the above-incorporated U.S. Patent Publication No. 2024/0148956. The filter is configured to separate blood from the undesired intravascular material, thereby allowing a user to visualize the thrombus captured, and to optionally reinfuse the blood back into the patient. By forming the reservoir or waste container of a clear material, it is possible for a user to visualize the filtered undesired intravascular material within the collection/waste container in real time to ensure that the material has been properly removed from the patient's body. The filtered bodily fluid collected in the container can optionally be reinfused back into the patient during the procedure, via the aspiration systems disclosed herein.

For instance, in another embodiment (not shown), instead of a waste assembly attached to the system for the removal and disposal of the undesired intravascular material and removed bodily fluid, the system can include a reinfusion assembly to filter the undesired intravascular material and return the filtered bodily fluid back to the patient. In this embodiment, the reinfusion assembly includes at least one filter and a reinfusion cannula. The filter is placed in fluid communication between cannula 130 and the reinfusion cannula. The filter can entrap and remove any debris from the undesired intravascular material, thereby filtering the bodily fluid removed from the patient in preparation for reinfusion. In one embodiment, the filter can be directly connected to the waste port, or a proximal end of an accessory cannula is attached to the waste port and a distal end of the accessory cannula is attached to the first side of the filter. A proximal end of the reinfusion cannula is then attached to a second side of the filter. A distal end of the reinfusion cannula is placed in, or placed in fluid communication with, the vasculature of the patient in a manner configured to reinfuse or return the filtered bodily fluid back to the patient. For instance, in one or more embodiments, the filtered bodily fluid can be reinfused through the introducer sheath assembly, such as described herein. An advantage of such an embodiment is the system can continuously and simultaneously aspirate, filter and reinfuse the bodily fluid back into the patient, thereby minimizing or reducing the risk of fluid loss and/or shock. For instance, because the filtered blood is simultaneously and continuously reinfused back into the patient, the risks associated with removing more than the total recommended volume of blood in a single procedure are minimized.

In one or more embodiments, side port 134 of cannula 130 also provides access to the treatment site for insertion and removal of accessory or ancillary devices, such as retrievers, secondary treatment devices, balloon catheters, angiographic catheters, embolic protection devices, wires and the like, or another device. For instance, side port 134 can be used to insert a secondary device, or a secondary suction cannula (e.g., secondary suction catheter comprising a second expandable funnel and a second cannula shaft with a smaller French size than cannula shaft 131) to aid in the removal of the undesired intravascular material through the cannula side port 134, if desired. In addition, this side port 134 can be used to deliver fluids such as saline, thrombolic agents, contrast media, and other medicine. The side port can include an ancillary port adapter and an ancillary port lumen extending from the port adapter to the cannula port inflow lumen. The ancillary port adapter can be a luer-type fitting with sealing element to prevent inadvertent introduction of air into the system through the ancillary port lumen, a quick connect style fitting, or any other fitting as known in the art.

As noted, one or more embodiments, cannula 130 includes proximal cannula connection hub 132, and elongate cannula shaft 131 defining one or more cannula lumen, and a cannula distal tip section. In one embodiment, the cannula distal tip section includes an expandable funnel 135. In another embodiment (not shown) the cannula distal tip section can include a non-expandable member. An aspiration fluid pathway between the treatment site and the barrel of the manual mechanical aspiration source 101 is established by operatively coupling the cannula connection hub 132 to the cannula port connector of the aspiration source 101.

In one or more embodiments, cannula shaft 131 includes a cannula lumen extending from the connection hub to the distal tip section. In one or more other embodiments, such as depicted in FIGS. 9A-9B, cannula shaft 131 can include one or more additional lumens, which can extend for a selected distance within and coaxial along the cannula shaft, such that the cannula can be a unitary or multi-layer structure. For example, the additional lumens can be used to gain access for a guidewire, secondary device, or other medical device to the treatment site, while simultaneously creating a suction force through the cannula lumen on the undesired intravascular material. In one embodiment, the cannula shaft can be reinforced for enhanced cannula pushability, trackability, and/or maneuverability during advancement through the cannula sheath and the vessel. Such reinforcement can include one or more stiffening elements positioned between and/or around individual shaft layers or embedded within a cannula shaft layer. Reinforcement elements can be in the shape of a coil, weave pattern or other patterns. The entire length or selected portions of cannula shaft 131 can be reinforced. By way of example only, the working length of the cannula can be from about 5 cm to about 200 cm to accommodate a range of vessel lengths.

In one or more embodiments, the distal tip section of the cannula shaft can be pre-shaped to form an angle or curve such that when unconstrained, the expanding funnel becomes offset from the shaft's longitudinal axis, such as shown in FIG. 1A-IC. The offset can be between 10 and 180 degrees. The shaped tip section profile can be formed through standard heat shaping techniques or by utilizing reinforcement elements such as described herein. The curved tip section can be advantageous when engaging an undesired intravascular material which is partially or fully attached to a vessel wall, and when the undesired intravascular material is located in tortuous or difficult to reach vasculature, such as a heart chamber or in a pulmonary vasculature.

In one or more embodiments, the cannula distal tip section includes an expandable funnel 135 for engaging and moving the undesired intravascular material into a lumen of cannula shaft 131. The structural aspects of funnel 135, including length, profile, structure, and flexibility can be designed to maximize clot retrieval while minimizing vessel damage. Funnel 135 can have an unexpanded or compressed configuration and an expanded configuration. When in an unexpanded state, funnel 135 can have an outer diameter roughly equivalent to the diameter of cannula shaft 131. In the expanded configuration, funnel 135 can form a substantially conical shape (in one embodiment) with the distal most funnel opening having a diameter larger than the cannula shaft diameter. In one embodiment, the diameter of the funnel opening when fully expanded can be about 14 mm. The diameter of the funnel 135 can be dictated by the diameter of the target vessel. For example, various sized cannula shafts 131, including varying sized funnels 135 can be selectively used in combination with aspiration system 100. The wall of funnel 135 can be formed from the cannula shaft 131, or can be comprised of an impermeable or semi-impermeable material. Funnel 135 can be self-expanding or mechanically actuated. In one example, the funnel 135 deploys when the funnel 135 is advanced past the distal end of the cannula sheath shaft 121 or the cannula sheath shaft 121 is pulled proximally relative to the cannula shaft 131 such that the funnel 135 escapes out the distal end of the cannula sheath shaft 121. In one embodiment, funnel 135 can include a plurality of expandable and independent struts or arms, encased, or otherwise attached to a semi-impermeable or impermeable membrane layer. Several embodiments of one or more aspects of a cannula or cannula assembly such as noted herein are described in more detail in the above-incorporated US Patent Publication No. 2020/0164117 A1.

As noted, in one or more embodiments, aspiration system 100 also includes cannula sheath assembly 120 and introducer sheath assembly 110. In one or more embodiments of aspiration system 100, sheath assemblies 120, 110 are operatively assembled such as depicted in FIGS. 1A-1B, with cannula sheath assembly 120 disposed, in part, between cannula 130 and introducer sheath assembly 110. As noted, cannula sheath assembly 120 includes a cannula sheath connection hub 122 (or first hub), and introducer sheath assembly 110 includes an introducer sheath connection hub 112 (or second hub). In one or more embodiments, connection hubs 122, 112 can be similar connection hubs, with dimensions of one or more components varying as needed to accommodate the respective elongate sheaths, or shafts. In one or more embodiments, the cannula sheath connection hub 122 and cannula sheath are sized and configured to accommodate the cannula 131, and the introducer sheath connection hub 112 and introducer sheath are sized and configured to accommodate the cannula sheath 121. When coaxially arranged as illustrated in FIGS. 1A-1B, cannula 130, cannula sheath assembly 120 and introducer sheath assembly 110 facilitate insertion and advancement of cannula 131, as well as the cannula sheath 121 and introducer sheath 131 into a vessel of a subject. Note that a variety of aspiration system configurations, alterations, and uses are possible, such as described herein.

As illustrated, the cannula sheath hub 122 and introducer sheath hub 112 are proximal hubs for the respective sheath assemblies, and in the depicted embodiment, each hub 122, 112 includes a side port 125, 115, respectively. In one or more embodiments, side ports 125, 115 can facilitate, for instance, a saline flush and/or blood return/reinfusion (e.g., via a syringe (not shown)) inserted into the side port, for instance through a one or more openings in the side wall of introducer sheath 111 near the distal end, such as illustrated in FIG. 3B.

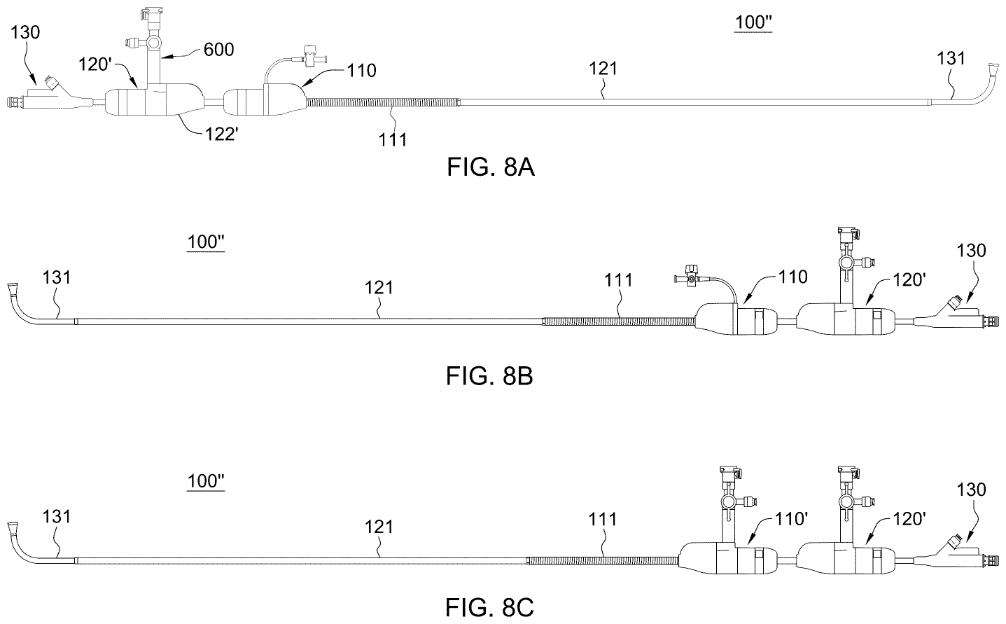

As noted, in the embodiment of FIGS. 1A-4B, the connection hubs 122, 112 of sheath assemblies 120, 110 are similarly configured, and aspiration system 100 includes introducer sheath assembly 110, cannula sheath assembly 120, and cannula 130, in one example only. This configuration of aspiration system 100 is advantageous where cannula sheath assembly 120 is used for hemostasis. For instance, aspiration system 100 can be employed when a user does not wish to do aspiration via the cannula sheath assembly side port, and thus, the side port configurations of the FIGS. 1A-4B can be used, as opposed to a large-bore side port configuration of the side port, such as described further below with reference to FIGS. 6A-8B. In such an embodiment, the cannula sheath assembly 120 can be used to deploy the funnel 135 of the cannula 130 as described elsewhere herein. Note also that, depending on the aspiration system configuration and the procedure, the use of cannula 130 can be optional. For instance, in one or more embodiments, cannula 130 of aspiration system 100″ of the embodiment of FIGS. 8A-8B can initially be used to remove a portion of the undesired intravascular material, and then removed to allow for removal of another portion of the material through the cannula sheath 121, such as described herein.

FIG. 4C shows another embodiment for providing large-bore aspiration via the cannula sheath 120. In this embodiment, a connector 420 is provided and configured to insert within the proximal end of the hub 122 to provide a bridge from the proximal end of the hub 122 to cannula shaft 121. The connector 420 can include a proximal port 422 configured to be operatively coupled to an aspiration source such as a syringe or manual mechanical aspiration device, such as any of the manual mechanical aspiration devices described herein. The connector 420 can include a shaft 424 configured to be inserted within the hub 122 of the cannula sheath 120. The shaft 424 can have a length sufficient to cross through the scaling gland and cross-slit valve of the hub 122. A distal end 426 of the shaft 424 is configured to matingly engage and/or abut with the shaft 121 of the cannula sheath 120 within the hub 122. In some embodiments, the interface of the connector shaft 424 and cannula sheath shaft 121 can include a press fit, snap fit, a seat, an o-ring, a threaded connection, and/or any now known or later developed connection for fluidly coupling the connector shaft 424 and cannula shaft 121. This embodiment of the disclosure provides for large-bore aspiration using the cannula sheath 120 without cannula 130 present, while providing a slimmer profile design for the sheath hub 122 given that a smaller bore side port 125 is provided without the need for a large-bore side port or large-bore side port 600 (FIG. 6A-8B), which has a larger profile and may be cumbersome in certain scenarios.

As discussed, in one or more embodiments, the connection hubs 122, 112 each include a hemostasis valve with a sealing mechanism, such as first multi-durometer scaling gland 310 (FIG. 4B) and second multi-durometer scaling gland 310′ (FIG. 3C), respectively. The seals, or sealing glands, which are described further below, advantageously prevent fluid backflow, such as by forming an enhanced seal around the respective shaft passing through the connection hub. In one or more further embodiments, connection hub 122 and/or connection hub 112 can include a mechanism to lock and unlock the position of cannula shaft 131 or cannula sheath 121, respectively, relative to the cannula sheath 121 or introducer sheath 111. For instance, when connection hub 122 is unlocked, cannula shaft 131 can be longitudinally moved relative to cannula sheath 121 and repositioned relative to the undesired intravascular material. Similarly, when connection hub 112 is unlocked, cannula sheath 121 can be longitudinally moved relative to introducer sheath 111 and repositioned, for instance, relative to the undesired intravascular material. In one or more embodiments, the connection hubs can be based on a Tuohy-Borst adapter/fitting or other known adapter/fitting, modified as described herein.

By way of specific example, and as illustrated in FIG. 4B, the connection hubs, such as connection hub 122 of cannula sheath assembly 120, can include a trim cone 400 and a distal housing 402 with a base 404 secured thereto via one or more torque screws 406. A mid-body 408 accommodates a lever 410 which, in one or more embodiments, is configured to allow a user to selectively apply a compressive force to multi-durometer scaling gland 310 about the cannula shaft 131, when the cannula shaft 131 extends coaxially through a central opening in connection hub 122 and the sealing gland, such as illustrated in FIGS. 1A-1B. In one or more embodiments, torque screws 406 further secure a proximal housing 412 with a central funnel shaped opening to mid-body 408, base 404 and distal housing 402. In one or more embodiments, lever 410 is configured to selectively apply pressure against multi-durometer sealing gland 310 to tightly seal the structure about the respective shaft, or to close completely where there is no shaft or other device passing longitudinally through the connection hub. As illustrated, side port 125 is in fluid communication with the coaxial inner chamber of the connection hub, which also includes a cross-slit valve 414 designed to open at a predetermined pressure, and to facilitate backflow prevention.

Depending on the procedure, such as during a thrombectomy procedure, a user may prefer to leave a guidewire in place (for instance, to be able to work over the wire) during the procedure for patient safety, navigation, maintaining access, and device exchange purposes. With current thrombectomy devices, maintaining hemostasis can be an issue when smaller coaxially placed secondary devices, such as a guidewire or angiographic catheter, are coaxially placed in the lumen of the thrombectomy device. For instance, the commonly known Tuohy-styled valve has a small gap remaining at maximum compression, and the cross-slit valve has no internal compressive ability, so that the coaxially placed secondary device opens a flange creating a window for fluid passage. In another example, the cross-slit valve of a conventional thrombectomy device can have bossed letters on the back of the valve that can rest on an injector pin marking of a hub or housing. In certain orientations, a leak path can thus be created during use.

In one or more aspects, improved hemostasis valves are disclosed herein for use with aspiration systems such as described herein, including with manual mechanical aspiration (MMA) thrombectomy devices, to reduce blood loss during a medical procedure, such as when the secondary device or an aspiration cannula is coaxially placed through a lumen of the aspiration system. For instance, the improved hemostasis valves, with multi-durometer seals such as described herein, are configured to maintain hemostasis during a variety of clinically relevant scenarios including, for instance, where no secondary device is coaxially inserted through the sealing gland and the lumen of the connection hub (0 Fr), and where a guidewire, such as known in the art, is coaxially inserted through the connection hub scaling gland and lumen of the aspiration system (where such guidewire is known in the art to typically have a diameter of 0.014″-0.035″), or where an angiographic catheter, such as known in the art, is coaxially inserted through the connection hub and lumen of the aspiration system (where such angiographic catheters typically have an outer diameter of 5 Fr-9 Fr), or where one or more secondary devices or other procedure-related medical components (cannula, obturator) are to be used, and are configured to be placed through the connection hub and lumen of the aspiration system. Additionally, the improved hemostasis valves of aspiration systems such as disclosed herein are configured to allow passage of a cannula, or cannula sheath, depending on the connection hub, or other medical device, that can have an expandable portion, a portion that is non-circular in shape, or includes an outer diameter up to about 21 Fr, by way of example only. Further, in one or more aspects, the improved hemostasis valve disclosed herein for a connection hub of an aspiration system is configured to provide an appropriate closing force during valve compression. For example, the improved hemostasis valve disclosed does not provide too much force as to crush or unintentionally reduce the diameter of an open lumen of the secondary device (e.g., cannula sheath or cannula) coaxially placed therethrough. However, the hemostasis valve also needs to provide sufficient force to not unintentionally unlock during use.

In one or more embodiments of a connection hub, the proximal body or housing includes a ¼ turn funnel, the mid-body includes a ¼ turn mid-body, the lever includes a ¼ turn lever, and a sliding washer, such as a ¼ turn sliding washer, is disposed between the lever and the multi-durometer sealing gland. In one embodiment, the sealing gland resides within the base, which can be a ¼ turn base, in one example. Note that the ¼ turn configuration of certain proximal hub components represents one example only.

In one or more aspects, the multi-durometer sealing gland in the connection hub of the cannula sheath assembly and/or introducer sheath assembly can include, in one embodiment, an intermediary layer disposed between outer layers, where the intermediary layer has a durometer different from a durometer of at least one outer layer of the outer layers. For instance, the durometer of the intermediate layer can be lower than the durometer of the at least one outer layer. In one or more embodiments, each outer layer can have the same or different durometers than the other outer layer. In one or more implementations, the multi-durometer sealing gland is a duo-durometer sealing gland, where the intermediate layer has a durometer lower than the durometer of the outer layers between which the intermediary layer resides. In one or more implementations, the improved hemostasis valve of one or more of the connection hubs of the aspiration systems includes a multi-durometer sealing gland that is capable of closing down to zero and/or completely closing, for example, on a guidewire.

In one or more embodiments, the multi-durometer sealing gland includes three layers, where the intermediary layer has a lower durometer than the two outer layers between which the intermediary layer resides. In such an embodiment, the lower durometer intermediary layer is softer, more compressible, and therefore, can close around a guidewire or angiographic catheter more readily to prevent any leaks during a procedure. The high-durometer outer layers further provide a stronger overall seal that will better remain in place within the connection hub. Note that in one or more implementations, the multi-durometer sealing gland can be the same in both the cannula sheath connection hub and the introducer sheath connection hub, or different.

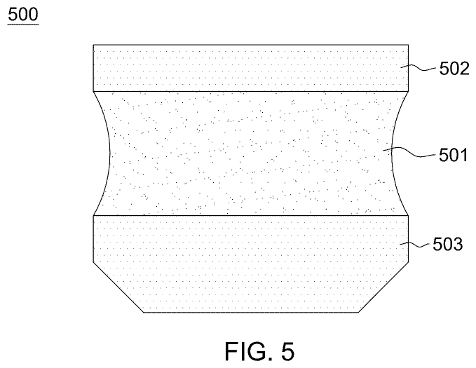

FIG. 5 illustrates one embodiment of a multi-durometer sealing gland 500, such as multi-durometer sealing gland 310 or multi-durometer scaling gland 310′ described above, where intermediary layer 501 has a lower durometer than outer layers 502, 503 between which intermediary layer 501 resides. FIG. 5 illustrates, by way of example, one embodiment of a dual-durometer sealing gland where the outer layers have the same durometer, which is different from the durometer of intermediary layer 501. As noted, the improved hemostasis valve, and in particular, the multi-durometer sealing gland of the hemostasis valve can be used in any connection hub, or multiple connection hubs of the aspiration systems, in accordance with one or more aspects of the present disclosure.

By way of further explanation, in a dual-durometer sealing gland embodiment, the scaling gland can be fabricated of silicone and include a first layer, second layer and third layer, with the second layer sandwiched or positioned between the first and third layers, such that the first and third layers are the outer layers, and the second layer is the intermediate layer. In certain embodiments, the first and third layers can have a first durometer, and the second layer can have a second, lower durometer. For example, in one embodiment, the first and third layer can each have a durometer of about 50 A to about 70 A, while the second layer can have a durometer of about 5 A to about 10 A. In one example, the first and third layers can each have a durometer of approximately 50 A, while the second layer has a durometer of about 5 A. In another example, the first and third layers can each have a durometer of approximately 70 A, while the second layer has a durometer of about 5 A. In such embodiments, the lower durometer second layer is softer, more compressible, and therefore, can completely close, or can more readily close around a guidewire or angiographic catheter to prevent any leaks. Also, the high durometer first and third layers result in a stronger overall sealing gland 500 that will better remain in place within the connection hub.

In yet another embodiment, the third layer can have a durometer of about 50 A-70 A, while each of the first and second layers can have a durometer of about 5 A. In such an embodiment, the first and second layers can be formed together as a single layer, resulting in multi-durometer, duo-layer sealing gland, as opposed to a multi-durometer, tri-layer scaling gland discussed above. Such an embodiment can reduce torque due to more of the sealing gland being composed of softer material. In one or more embodiments, the sealing gland can have a larger or thicker inner portion that more readily clamps under compression onto the guidewire or catheter passing through a central opening in the sealing gland, resulting in a larger contact surface area, and stronger seal.

Referring to the multi-durometer, tri-layer embodiment of FIG. 5, the second layer or intermediary layer 501, can include a substantially concave circumferential surface as illustrated, to aid in inwardly directed compressibility of the second layer around a cannula, cannula sheath, guidewire or angiographic catheter, or other device, that may be inserted through the connection hub within which the hemostasis valve resides. Additionally, as illustrated in FIG. 5, in one or more embodiments, one of the outer layers, such as outer layer 503 can include a tapered end while the other outer layer can include a substantially flat base surface. The tapered end of outer layer 503 is configured to matingly engage a tapered cavity within base 404 (FIG. 4B), such as a ¼ turn base. The flat base surface of the first outer layer 502 results in a uniform compression by the washer 407 mating therewith in the connection hub embodiment discussed herein.

In one or more other embodiments, the third layer or outer layer 503 can have a durometer of about 70 A, providing a harder bottom layer that would be more difficult to push the sealing gland through the quarter turn base 404 (FIG. 4B), and thereby result in the scaling gland remaining in position more reliably. Only the intermediary layer 501 or second layer, gets compressed, thereby resulting in a more repeatable and/or reliable distance for lever 410 (FIG. 4B) to turn for closure. If the sealing gland is pushed out, then the zero point moves as the scaling gland moves. This can also reduce torque, as only compressing of the softest portion of the sealing gland is required, instead of losing energy pushing the harder part out of the base cavity.

In another embodiment where the top outer layer 502 has a durometer of about 5 A, the torque can be further reduced by the presence of the softer material. Also, as noted, having a larger or thicker intermediary layer that clamps onto, for instance, the wire or catheter results in a larger contact surface, and thus, a stronger seal. The sealing gland 500 can be made by a two-shot molding process (i.e., overmolding, dual-shot, or multi-shot molding). For example, in a first phase of the molding process, the outer layers 502, 503 can be formed with a spacer positioned therebetween such that an empty space is formed between the outer layers 502, 503 during the molding. In a second phase of the molding process, the empty space is then filled in with the intermediary layer material 501. In some embodiments, the mold can be sandblasted such that peaks and valleys or small ridges or bumps are formed on the sealing gland 500 thereby creating non-contact surfaces.

As illustrated in FIG. 4B, in one or more embodiments, lever tooth 409 and washer 407 tooth heights can be in a common range, with the multi-durometer sealing gland being configured to reside between washer 407 and base housing 404. In one or more embodiments, the assembled stack up of the connection hub can result in a pre-set interference (with level at full rest position). When the torque screw(s) is positioned and tighten within the torque screw opening, some compression on the multi-durometer sealing gland is provided. When the ¼ turn lever is turned, additional compression is provided on the sealing gland so that the sealing gland closes down on the instrument passing through the multi-durometer sealing gland, within the connection hub, thereby providing hemostasis. In one or more embodiments, an engagement feature can also be provided between the lever and the washer (that is between the ¼ turn lever and the ¼ turn washer), where the two engage at the slanted surface of the ¼ turn washer tooth. More specifically, the engagement feature can include a mating bump and/or a detent configuration that aids in locking and overall security of the connection between the washer and lever. In one or more other embodiments, a washer or O-ring, such as silicone washer or O-ring, can be added to the connection hub behind the cross-slit valve 414 (FIG. 4B), between cross-slit valve 414 and the ¼ turn base 404.

As noted, multiple enhanced aspiration systems and aspiration system aspects, are disclosed herein. By way of further example, FIGS. 6A-6C collectively illustrate an aspiration system 100′, which unless otherwise indicated, is the same as, or similar to, aspiration system 100 described above in connection with FIGS. 1A-5. Aspiration system 100′ of FIGS. 6A-6C further addresses several issues with existing aspiration system designs. Specifically, certain connection hub designs cannot be used for aspiration due to small-bore size arm tubing and the presence of a stopcock with luer terminal connections.

In the aspiration system 100′ embodiment of FIGS. 6A-6C, a cannula sheath connection hub 122′, similar in certain aspects to cannula sheath connection hub 122 of aspiration system 100, is depicted as including a side port with a large-bore side port 600 configuration that includes a large-bore tubing 601 extending from the side of the connection hub, as well as a stopcock 610, such as a two-way or three-way stopcock, and a quick connect 620. This large-bore side port 600 provides a user with the ability to connect to a manual mechanical aspiration source or aspiration device, such as manual mechanical aspiration source 101 described above in connection with FIGS. 1A-4B. For instance, this provides an ability to connect a manual mechanical aspiration source to the cannula sheath connection hub, creating a large-bore aspiration pathway from the distal end of the cannula sheath to the aspiration source coupled to the large-bore side port 600 on the side of the connection hub. Note that in the embodiment of FIGS. 6A-6C, there is no cannula, such as cannula 130 (FIGS. 1A-1B), in the operative assembly of aspiration system 100′. In this configuration, aspiration is through the cannula sheath assembly 120, and in particular, through the large-bore side port 600, which is in fluid communication through connection hub 122′ with cannula sheath 121, and in particular, the lumen of cannula sheath 121. Note that in the configuration of FIGS. 6A-6C, stopcock 610 includes a twist lever on one side to allow for aspiration through large-bore side port 600, or to allow the large-bore side port to be used, for instance, for injecting a flush or a contrast through the cannula sheath into the vessel of the patient. Note also that a variety of device designs can be used in association with large-bore side port 600 to close the side port when desired, such as, for instance, one or more tethered end caps, stopcocks, clamps, etc.

A clinical advantage of aspiration system 100′ is that it provides a secondary method of aspiration for clinical situations where the compliant funnel may be non-beneficial or provides a bridge during a procedure where the funnel can be properly utilized. Additionally, aspiration system 100′ of FIGS. 6A-6C can simplify visualization during a procedure, aid in vessel access through the cannula sheath, reduce procedure cost for a patient, and/or aid in aspiration of smaller vessels prior to cannula insertion. As noted, with large-bore side port 600 of cannula sheath connection hub 120′, side port aspiration can be performed through cannula sheath 121 using a manual mechanical aspiration source or device, such as manual mechanical aspiration source 101 of FIG. 1A. In this configuration the cannula has been removed from the cannula sheath, and large-bore side port 600 allows for large clots (such as clots that are approximately the size of the inner diameter of the sheath) to be removed, while stopcock connection 610 allows for closing off of the fluid path, and holding of an aspiration pressure, such as, for instance, up to a pressure of 14 psi. Note also that in this configuration, the hemostasis valve (described above) of cannula sheath connection hub 120′ is actuated to completely seal the multi-durometer sealing gland against loss of fluid through the proximal end of the connection hub.

In one or more embodiments, the distal housing of cannula sheath connection hub 120′ is sized and configured to accommodate the large-bore port for side port 600. Large-bore tube 601 can be fluidly connected to the large-bore port of the cannula sheath connection hub to facilitate side port aspiration. Opposite the large-bore port can be a quick connect or other connection known in the art, for fluidly connecting the large-bore side port to an aspiration source. As noted, a stopcock 610 can be provided in tube 601 for controlling aspiration from the aspiration source. With the stopcock, a flush port 611 or luer port or swappable luer port, can also be provided to, for instance, facilitate fluidly coupling a flushing source via the large-bore side port and cannula sheath connection hub 120′ to the cannula sheath.

Note that in one or more other embodiments, cannula sheath 120′ can include a funnel insertion tool or other secondary device that can be used to aid coaxial movement of an aspiration catheter with an expandable distal end through the connection hub and the lumen of the cannula sheath 121. For example, the cannula sheath can include a relatively large-bore channel or lumen extending from and partially within the distal housing cavity. Open space within the distal cavity can allow for an expandable distal end of an aspiration catheter, such as an expandable funnel shaped distal end, to partially expand and/or a reshaped curve of the aspiration catheter to form, which can catch a lip of the sheath channel or lumen and not pass into the cannula sheath shaft. In such a case, a funnel insertion tool or other secondary device can be provided to aid in compressing the distal end of the aspiration sheath (i.e., with an expandable funnel) for entrance into the cannula sheath.

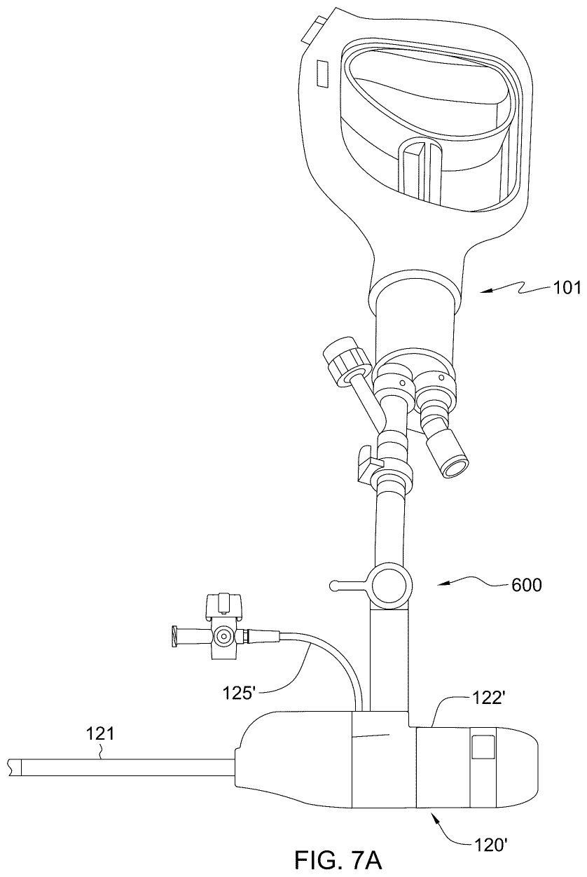

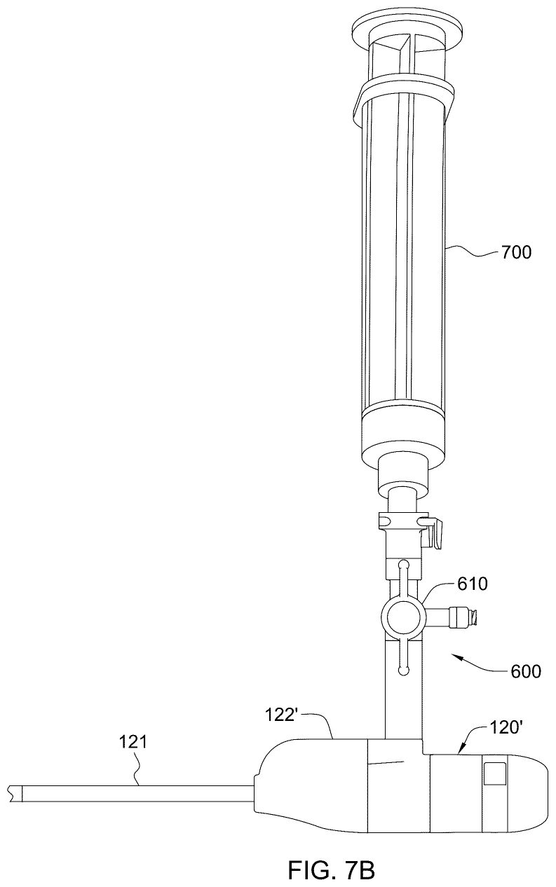

As noted, a variety of aspiration system configurations are disclosed herein. In one or more other embodiments, two ports can be included in the cannula sheath connection hub. For example, as illustrated in FIG. 7A, a large-bore side port 600 such as illustrated in FIGS. 6A-6C can be included for connection of a manual mechanical aspiration source 101 to the large-bore side port, such as described above, as well as another side port 125′ for injecting a flush into the cannula sheath 121, where desired. In one or more embodiments, side port 125′ can terminate in a scalable connection, such as a luer port. Additionally, in one or more embodiments, a clamp can be provided along the length of the large-bore side port to allow for control of aspiration from a manual mechanical aspiration source 101 to the cannula sheath connection hub 122′. As noted, in one or more other embodiments, a two-way, or a three-way, stopcock valve can be provided, such as the three-way valve illustrated in the embodiment of FIG. 7B, where a syringe is alternatively illustrated as aspiration source for an aspiration procedure through the cannula sheath assembly 120′.

In one or more embodiments, a tethered endcap (not shown) can be provided in association with large-bore side port 600. For instance, the tethered endcap can be tethered to an end of tube 601 and include a ring or connection element that is configured to connect to and/or sit on the tube, with the endcap being removably inserted within the lumen of the tube, and the tether coupling the connection element in the endcap.

In yet further aspiration system embodiments, a valve can be bonded within the distal housing of the cannula sheath connection hub that does not require any tubing. In one embodiment, the flushing source can be directly fluidly coupled with the bonded valve to provide flushing into the cannula sheath. This embodiment of the cannula sheath assembly and aspiration system is compatible with either the clamp or tethered clamp for controlling aspiration through the large-bore side port from the aspiration source.

In yet another embodiment of cannula sheath aspiration, a T-port can be positioned in line with the large-bore side port to the aspiration source. In this embodiment, the T-port can include a flushing port for directly fluidly coupling to a flushing source. This embodiment is compatible with either a clamp or tethered cap (not shown) for controlling aspiration through the large-bore side port tube from the aspiration source. Further details of these aspects of one or more of the embodiments are described, by way of example, in the initially incorporated provisional applications. As noted in the art, there are times when a manual mechanical aspiration source, such as manual mechanical aspiration source 101 (FIG. 1A), cannot be used for infusion. Similarly, there can be issues with sheath aspiration and same connection contrast injection. There is a need in the art for the ability to also connect to a side port, such as a cannula sheath large-bore side port 600, in order to deliver a large volume quickly using, for instance, a 60 cc syringe with a large-bore opening. Advantageously, the syringe does not require excessive force for hand injection, and/or is able to be used to inject fluid without leaking. An example of this assembly depicted in FIG. 7B, where a large-bore syringe is used in combination with large-bore side port 600 in order to inject fluid through the cannula sheath connection hub 122′ into the cannula sheath, as desired.

By way of further example, FIGS. 8A-8B illustrate another embodiment of an aspiration system 100″ which is the same as or similar to aspiration systems 100, 100′ described above in connection with FIGS. 1A-7B. In this embodiment, cannula sheath assembly 120 of aspiration system 100 of FIGS. 1A-4B is replaced by cannula sheath assembly 120′ of FIGS. 6A-7B. In this configuration, a user is provided with an option to aspirate through either cannula 130, such as described above in connection with FIGS. 1A-4B, or through large-bore side port 600 of cannula sheath connection hub 122′ when cannula 130 is removed, such as described above with reference to FIGS. 6A-7B. In such an embodiment, it can be desirable to aspirate at least a first portion of the undesired material through cannula 130, and then remove the cannula 130 from the cannula sheath assembly 120′ to aspirate at least a second portion of undesired material through cannula sheath assembly 120′, for example where needed to capture larger portions of undesired material incapable of being captured by the cannula 130. In some embodiments, it can be desirable to aspirate first through the cannula 130 and then withdraw the cannula 130 from the cannula sheath 120′. Once the cannula 130 is removed, an aspiration can be performed through the cannula sheath 120′ to remove any debris of the undesired material that may have entered the cannula sheath 120′. Once cleared, the cannula 130 can be reintroduced to the cannula sheath 120′ and additional aspirations can be performed with the cannula 130. This process can be repeated as may be deemed necessary by the clinician. In another embodiment, it can be desirable to aspirate at least a first portion of the undesired material through cannula sheath assembly 120′ and then insert cannula 130 into cannula sheath assembly 120′ to aspirate at least a second portion of undesired material through the cannula 130. For example, when treating deep vein thrombosis (DVT), a clinician may access the DVT from behind the knee to the popliteal. In such a clinical scenario, aspirating through the larger cannula sheath 120′ first can create a landing zone for the smaller cannula 130 thereby making it easier to deploy the funnel 135. When treating a PE, a clinician can position the cannula sheath 120′ within the main pulmonary artery before its branches to the left and right lungs (e.g., the saddle of the pulmonary artery). The cannula sheath 120′ can be used to aspirate large portions of the undesired material within the main pulmonary artery and then the smaller cannula 130 can be inserted with the cannula sheath 120′ and advanced within the smaller branches to remove any undesired material within the smaller branches.

As another example, FIG. 8C illustrates another embodiment of aspiration system 100″, which is the same or similar to aspiration systems 100, 100′ described above in connection to FIGS. 1A-7B. In this embodiment, cannula sheath assembly 120 of aspiration system 100 of FIGS. 1A-4B is replaced by cannula sheath assembly 120′ of FIGS. 6A-7B to provide a large-bore side port into the cannula sheath connection hub. Similarly, in the embodiment of FIG. 8C, the introducer sheath assembly 110 of FIGS. 1A-4B is modified with a large-bore side port, similar to the large-bore side port for the cannula sheath assembly described herein. In this configuration, users are provided with an option to aspirate through, for instance, cannula 130, or through either of the large-bore side ports of the cannula sheath connection hub or introducer sheath connection hub, such as described herein. In one or more embodiments, it can be desirable to generate a suction force through the introducer sheath assembly 110 when, for example, debris of the undesired material become lodged or stuck within the introducer sheath 111 during aspiration of the undesired material via the cannula 130 or cannula sheath assembly 120. A suction force can be generated within the introducer sheath assembly 110 when the cannula 130 and cannula sheath assembly 120 are removed therefrom.

As noted, FIGS. 9A-9B illustrate one embodiment of a collapsible lumen for use in association with a tube 900, such as a cannula shaft 131, cannula sheath 121, or introducer sheath 111, of an aspiration system such as disclosed herein. FIGS. 9A & 9B illustrate presence of a collapsible lumen 910 in open and closed positions, respectively. Collapsible lumen 910 operates as an ancillary lumen to main lumen 901 of tube 900. Note that one or more collapsible lumens 910 can extend for a selected distance within and coaxial along the tube such that the shaft or sheath can be a multi-layer structure. In one or more implementations, the collapsible lumen(s) can be used to gain access for a guidewire, secondary device, or other medical device to the treatment site, while simultaneously creating a suction force through lumen 901 on the undesired intravascular material. In one or more other embodiments, the additional, collapsible lumen(s) 910 can be used to inject a fluid, such as via a side port in fluid communication with the collapsible lumen, when not aspirating through main lumen 901.

Those skilled in the art will note from the description provided herein that a variety of aspiration systems are disclosed, each with one or more inventive aspects such as described. The aspiration systems can be used in a variety of ways depending on the desired procedure to, for instance, remove an undesired material from a circulatory system of a subject. Various methods of aspiration and removal of an undesired intravascular material are described below by way of example only

In one or more embodiments, methods for removing an undesired material from a vasculature are provided herein. For instance, in one or more embodiments, one or more methods includes providing an aspiration system with a distal end proximate the undesired material. The aspiration system includes one or more of the novel aspects described herein. For instance, in one embodiment, the aspiration system includes a first sheath assembly having a first elongate shaft and a first hub at a proximal end of the first elongate shaft. The first hub includes a hemostasis valve including a first multi-durometer sealing gland and a side port configured to be operatively coupled to a first suction source. In addition, the aspiration system includes a cannula and a second sheath assembly. The cannula includes a cannula shaft configured to be inserted within the first sheath assembly and to be operatively coupled to a second suction source, where the first multi-durometer sealing gland is configured to selectively seal around the cannula shaft or completely seal when the cannula shaft is not positioned within the first sheath assembly. The second sheath assembly includes a second elongate shaft having a tapered distal end and a second hub at a proximal end of the second elongate shaft. The second hub includes a hemostasis valve having a second multi-durometer sealing gland. The first elongate shaft of the first sheath assembly is configured to be inserted into the second elongate shaft of the second sheath assembly and the second multi-durometer sealing gland of the second sheath assembly is configured to seal around the first elongate shaft of the first sheath assembly.

In addition, in one or more embodiments, the method further includes actuating the second suction source to generate a suction force through the cannula shaft to remove a first portion of the undesired material from a vessel of the vasculature; removing the cannula shaft from the first sheath assembly; and actuating the first suction source to remove a second portion of the undesired material from the vessel of the vasculature via the first elongate shaft and the side port of the first hub of the first sheath assembly.

In one or more embodiments, the first suction source includes one of a syringe or a manual mechanical aspiration source. In addition, the second suction source can also include one of the syringe or the manual mechanical aspiration source. In one implementation, the first suction source and second suction source can each include a manual mechanical aspiration source, and in one or more other implementations, the first suction source and the second suction source can each include a syringe. In another embodiment, the first suction source and the second suction source can embody different types of suction sources with, for instance, one type being a syringe and the other type being a manual mechanical aspiration source.

In one or more embodiments, the tapered distal end of the second elongate shaft of the second sheath assembly includes a plurality of openings, and the method further includes reinfusing blood through the plurality of openings in the second elongate shaft of the second sheath assembly.

In one or more embodiments, the first multi-durometer sealing gland of the first hub seals against the cannula shaft with the second suction source actuated to remove the first portion of the material from the vessel through the cannula shaft, and is completely closed, or closed on a guidewire, or other device, with the cannula shaft removed from the first sheath assembly, and a first suction source actuated to remove the second portion of the undesired material from the vessel via the first elongate shaft and the side port of the first hub of the first sheath assembly.

In one or more embodiments, the side port of the first hub of the first sheath assembly includes an injection port, and the method further includes injecting a fluid or inserting a device through the injection port and the first elongate shaft into the vessel.

In one or more embodiments, a cannula hub of the cannula includes a side port coupled through the hub in fluid communication with the cannula shaft, and the method further includes injecting a fluid or inserting a device through the side port and cannula shaft of the cannula into the vessel. In one embodiment, the cannula shaft further includes a collapsible lumen, and injecting the fluid inflates the collapsible lumen to allow the fluid to flow through the vessel.

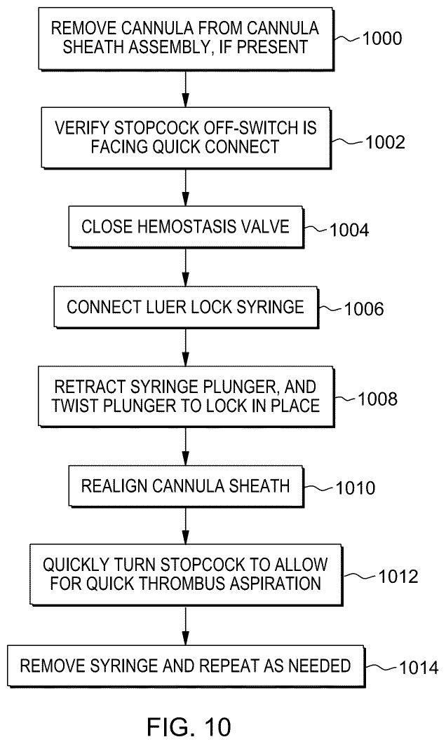

By way of further example, FIG. 10 depicts one embodiment of a method of aspiration via a cannula sheath side port of a cannula sheath hub of an aspiration system, such as depicted in FIGS. 8A-8B, in accordance with one or more aspects of present disclosure. As an example, the method of FIG. 10 is an exemplary method of aspiration via a quick bolus pull, where the aspiration source includes a syringe. Assuming normal system placement, navigation to the treatment area, and following a series of aspirations through the cannula, the method of FIG. 10 includes removing the cannula from the cannula sheath assembly, if present 1000, and verifying that the stopcock off switch is facing the quick connect 1002. The hemostasis valve may be completely closed by rotating the lever of the connection hub to prevent flow of fluid out the back of the cannula sheath connection hub 1004. A guidewire could still be in place, in which case, the lever could be turned far enough so that the multi-durometer sealing gland fully seals over and around the guidewire and/or remaining device. A luer lock syringe is connected 1006 to the quick connect port on the large-bore side port assembly. The syringe plunger can then be retracted and twisted so that the plunger fins rest on the locking flanges 1008 to lock the syringe plunger in place. Once accomplished, the syringe can rest without intervention in a state with negative pressure generated (i.e., pre-charged). The sheath can be realigned to target the desired treatment area, if necessary 1010. The method further includes quickly turning the stopcock to allow for quick thrombosis aspiration 1012, after which the syringe can be removed, and the process repeated as needed 1014.

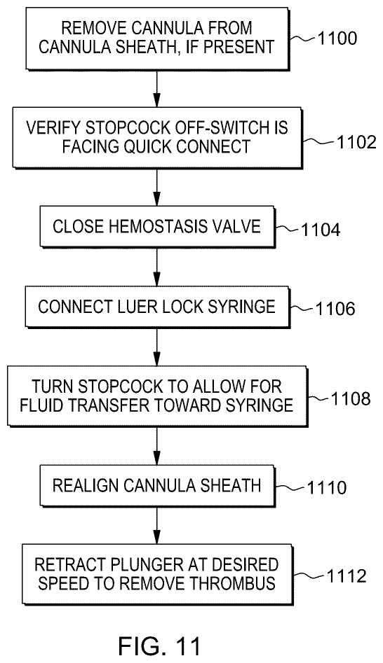

FIG. 11 depicts another embodiment of a method of aspiration via a cannula sheath side port of a cannula sheath hub of an aspiration system, such as depicted in FIGS. 8A-8B, in accordance with one or more aspects of present disclosure. The method of FIG. 11 depicts an exemplary method of aspiration via a hand-controlled device where the aspiration source includes, for instance, a syringe. Again, assuming normal system placement, navigation to the treatment area within the patient's circulatory system, and following a series of aspirations through the cannula, the method includes removal of the cannula from the cannula sheath, if present 1100, and verifying that the off switch on the stopcock is facing the quick connect 1102. The hemostasis valve is completely closed by rotating the lever on a connection hub to prevent fluid from leaking out the back of the cannula sheath assembly 1104. Should a guidewire or other device remain in place, the lever is turned far enough to compress the multi-durometer sealing gland over the wire and/or other device to form a fluid tight seal. The luer lock syringe is then connected 1106 to the quick connect port on the stopcock of the large-bore side port assembly, ensuring that the plunger is fully depressed (opposite of the placement described above). The stopcock connection is turned to allow fluid transfer toward the syringe 1108. The cannula sheath can be realigned to target the desired treatment area, if necessary 1110. The plunger can then be retracted at the desired speed to remove the thrombosis 1112, which as noted is, a hand-controlled method for removing the thrombosis.

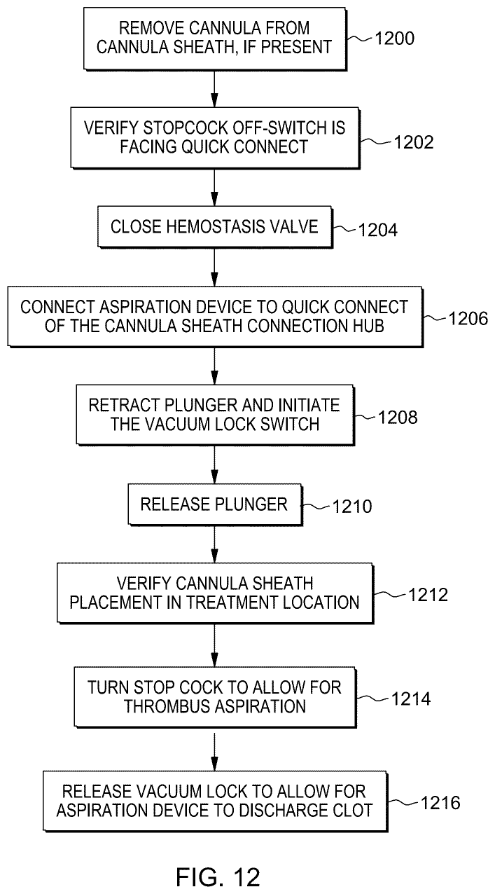

FIG. 12 depicts a further embodiment of a method of aspiration via a cannula sheath side port of a cannula sheath hub of an aspiration system, such as depicted in FIGS. 8A-8B, in accordance with one or more aspects of present disclosure. The method of side port aspiration assumes use of a manually-operated aspiration device 101 (FIG. 1A) by pre-charging aspiration. Further aspects of a manually-operated aspiration device, such as manual mechanical aspiration source 101 (FIG. 1A), are disclosed in the above-incorporated U.S. Pat. No. 11,648,020. Assuming normal system placement, navigation to the treatment area within the patient's circulatory system, and following a series of aspirations through the cannula, the method includes removing the cannula from the cannula sheath, if present 1200, and verifying that the stopcock off switch is facing the quick connect 1202. The hemostasis valve is completely closed by rotating the lever of the connection hub to prevent fluid leakage out the back of the cannula sheath assembly 1204. A manually-operated aspiration device, such as described herein, is connected to the quick connect of the cannula sheath 1206. The plunger of the aspiration device is retracted, and while fully retracted, the vacuum lock is initiated to maintain a position of the plunger, and thereby maintain the vacuum 1208. The plunger can then be released as the plunger is held in place by the vacuum lock 1210. The process can include verifying that the cannula sheath placement is in the desired treatment location 1212. The stopcock can be quickly turned to allow for quick thrombosis aspiration 1214. The vacuum lock can then be released to allow the aspiration device handle to discharge the clot into the waste line of the waste assembly for collection 1216. The process can be repeated as need for a particular procedure.

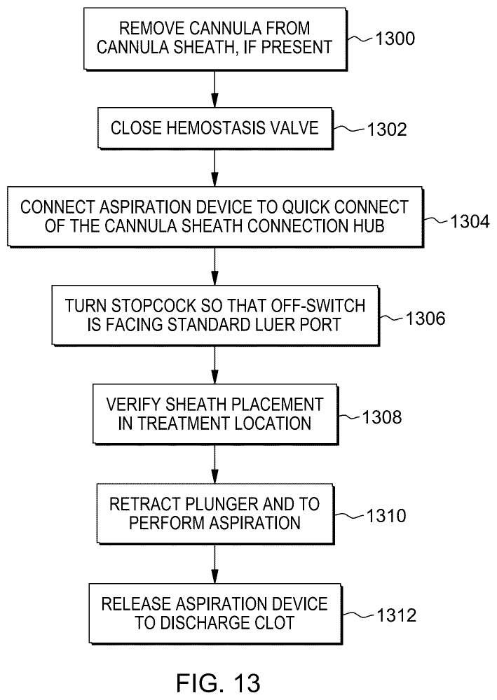

FIG. 13 depicts another embodiment of a method of aspiration via a cannula sheath side port of a cannula sheath hub of an aspiration system, such as depicted in FIGS. 8A-8B, in accordance with one or more aspects of present disclosure. In this embodiment, the method assumes, in part, side port aspiration using a manually-operated aspiration device to control aspiration, such as the above-discussed manually-operated aspiration device 101 (FIG. 1A), discussed further in the above-incorporated U.S. Pat. No. 11,648,020. Again, assuming normal system placement, navigation to the treatment area, and following a series of aspirations through the cannula, the method includes removing the cannula from the cannula sheath, if present 1300, and closing the hemostasis valve 1302. The method further includes connecting the aspiration device to the quick connect of the cannula sheath connection hub 1304, and verifying that the off switch on the stopcock is facing the standard luer port allowing for fluid travel to the activation device 1306. Further, the process can include verifying the cannula sheath placement with reference to the treatment location 1308. The plunger is retracted to perform an aspiration 1310. After retraction, the plunger can be released, to allow for the aspiration device to discharge the clot into the waste line of the waste assembly 1312. The method of FIG. 13 can be repeated as necessary as desired for a particular procedure.

Note that in one or more other embodiments, any of the methods described above in connection with FIGS. 10-13 can also be used, for instance, with aspiration system 100′ of FIGS. 6A-6C, with the first step of the method being omitted since the cannula is not used in that aspiration system embodiment.

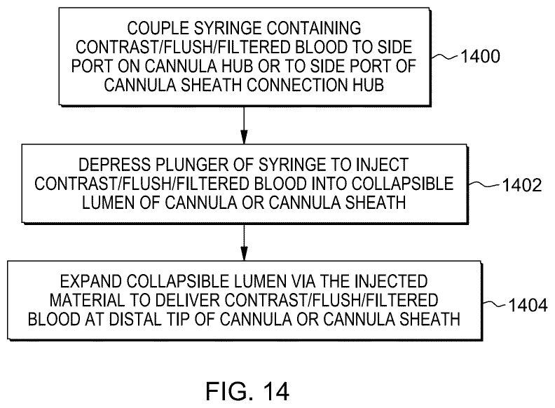

FIG. 14 depicts one embodiment of a method of injecting contrast, flush and/or filtered blood via a collapsible lumen of a cannula or cannula sheath of an aspiration system, such as depicted in FIGS. 1A-1C & 6A-9B, in accordance with one or more aspects of present disclosure. The method includes, in one embodiment, coupling a syringe containing contrast/flush/filtered blood to a side port on the cannula hub, or to a side port of the cannula sheath hub 1400, such as described herein. Once connected, the plunger of the syringe is depressed to selectively inject the desired fluid (e.g., contrast, flush, saline, thrombolic agents, medicine, or filtered blood) into the collapsible lumen of the cannula or the cannula sheath 1402. The collapsible lumen expands via the injected material to deliver the contrast/flush/or filtered blood at the distal end of the cannula or cannula sheath 1404 for injection into the vessel with the undesired material being treated. The collapsible lumen can be created using a PTFE liner, specialized mandrels and lamination processes. In some embodiments, the collapsible lumen can have a diameter of approximately 0.018 inches to approximately 0.035 inches, however, other dimensions are also contemplated by aspects of the disclosure. The collapsible lumen can provide the advantage of reducing the amount of contrast injected given the smaller lumen size.

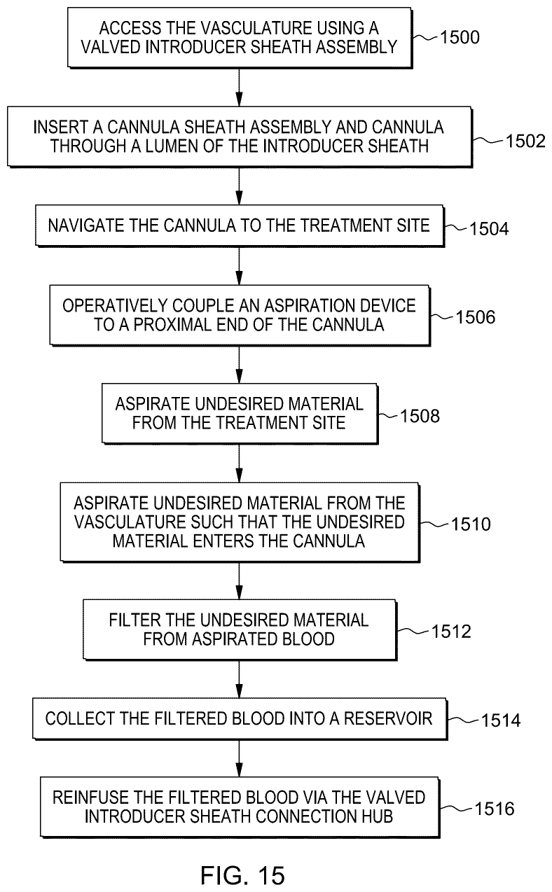

By way of further example, FIG. 15 depicts one embodiment of a method of reinfusion of blood through an introducer sheath of an assembled aspiration system, such as depicted in FIGS. 1A-C & 6A-8B, in accordance with one or more aspects of present disclosure. The method of FIG. 15 also illustrates another embodiment of a method of removing an undesired material from a treatment site. The method includes accessing the vasculature using a valved introducer sheath 1500, such as the introducer sheath assembly described herein. The vasculature can be accessed using known methods including using guidewires, dilators, and/or obturators to place the introducer sheath within the vasculature. A cannula sheath and cannula can be inserted into the valved introducer sheath assembly, such as through a lumen of the introducer sheath 1502. In one or more embodiments, the cannula can be inserted into the cannula sheath and an obturator can be inserted into the cannula, thereby coupling the cannula sheath, cannula, and obturator together. The coupled cannula sheath, cannula, and obturator can then be inserted into the introducer sheath, such that the cannula sheath, cannula, and obturator are positioned within the vasculature 1504. If a guidewire is still in place, the coupling of the cannula sheath, cannula, and obturator can be advanced over the guidewire, and once positioned, the guidewire can be removed. Once the cannula is navigated to the treatment site, the obturator can be removed, and an aspiration device can be operatively coupled to the proximal end of the cannula 1506. The cannula sheath can be pulled back to deploy a funnel at the distal end of the cannula. Alternatively, the cannula can be pushed forward such that the funnel is pushed out of the cannula sheath and deploys. The aspiration device can be activated to aspirate the undesired material from the treatment site 1508, with the aspirating continuing such that the undesired material enters the cannula 1510. Once the undesired material enters the cannula, it can pass through a filter, such as a filter described in the above-incorporated US Patent Publication No. 2024/0148956. The filter captures the undesired material, while allowing filtered blood to pass through the filter 1512. The filtered blood can be collected in a reservoir such as a waste container 1514. A syringe can then be used to collect and/or transfer the filtered blood from the reservoir. The syringe can be operatively coupled to a side port of the valved introducer sheath such that the filtered blood is transferred from the syringe and is reinfused into the patient via the side holes in the distal end of the introducer sheath 1516, such as described herein. In one or more embodiments, the reservoir can include a syringe, such that a separate reservoir to collect the filtered blood before being transferred to a syringe is not required.

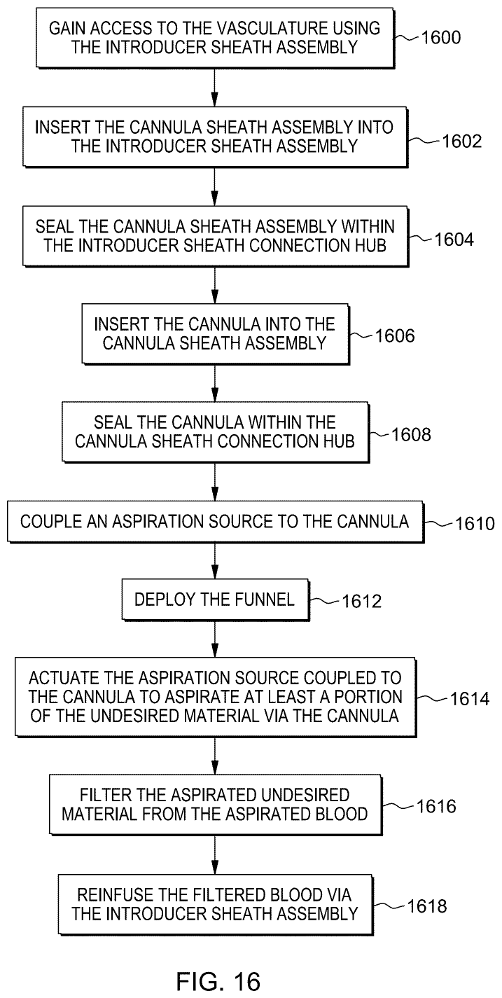

FIG. 16 depicts another embodiment of a method of removing, at least in part, an undesired material from a vasculature and reinfusing blood through an introducer sheath of an assembled aspiration system, such as depicted in FIGS. 1A-1C, in accordance with one or more aspects of present disclosure. The method includes accessing the vasculature using an introducer sheath 1600, such as the introducer sheath assembly described herein. The vasculature can be accessed using known methods including using guidewires, dilators, and/or obturators to place the introducer sheath within the vasculature. A cannula sheath is inserted into the introducer sheath assembly 1602, such as through a lumen of the introducer sheath. The method includes scaling around the cannula sheath assembly within the introducer sheath connection hub 1604 using, for instance, a multi-durometer scaling gland, such as second multi-durometer scaling gland 310′ (FIG. 4B). In one or more embodiments, the cannula is inserted into the cannula sheath assembly 1606, and a multi-durometer sealing gland (such as first multi-durometer scaling gland 310 (FIG. 4B)), is used to seal about the cannula within the cannula sheath connection hub 1608. In one or more embodiments, an aspiration source or device is operatively coupled to the proximal end of the cannula 1610 and the funnel at the distal end of the cannula is deployed 1612. The funnel can be deployed in a variety of manners. For instance, the cannula sheath can be pulled back to deploy the funnel at the distal end of the cannula. Alternatively, the cannula can be pushed forward such that the funnel is pushed out of the cannula sheath and deploys. The aspiration device or source is activated to aspirate at least a portion of the undesired material from the treatment site via the cannula, with the aspirating continuing such that at least the portion of the undesired material enters the cannula 1614. After the undesired material enters the cannula for removal, it can pass through a filter, such as a filter described in the above-incorporated U.S. Patent Publication No. 2024/0148956. The filter captures the undesired material, while allowing filtered blood to pass through the filter 1616. The filtered blood can be collected in a reservoir and then reinfused during the method via the introducer sheath assembly 1618, such as described herein. In one or more embodiments, a syringe can be used to collect and/or transfer the filtered blood from the reservoir. The syringe can be operatively coupled to a side port of the introducer sheath such that the filtered blood is transferred from the syringe and reinfused into the patient via the side holes in the distal end of the introducer sheath, such as described herein. Note that during the process of FIG. 16, fluids can be injected at any time deemed appropriate by a clinician via, for instance, the hub side ports 125, 134 (FIG. 1A). In addition, secondary devices can be deployed at any time during the process deemed appropriate by the clinician via the hub side ports 125, 134 (FIG. 1A).

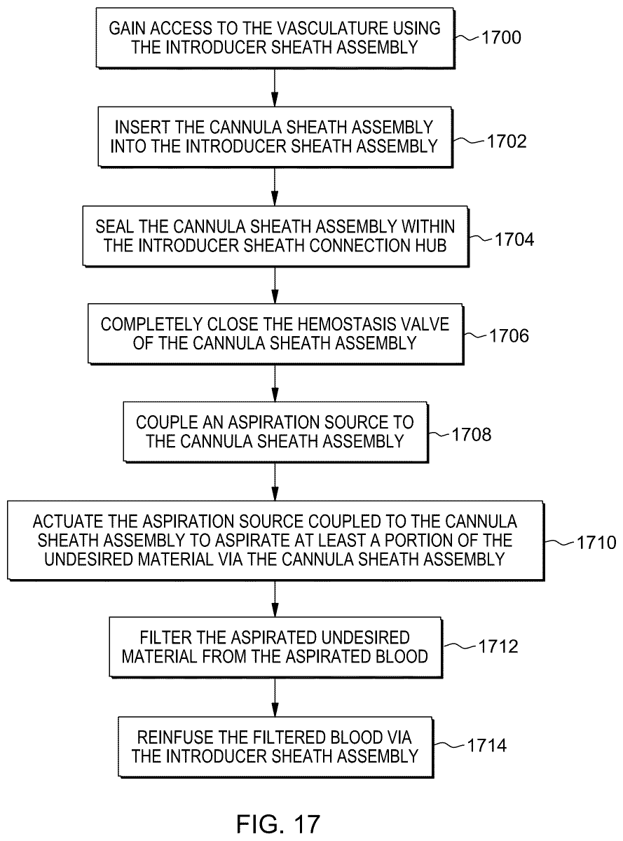

As a further example, FIG. 17 depicts another embodiment of a method of removing, at least in part, an undesired material from a vasculature and reinfusing blood through an introducer sheath of an assembly aspiration system, such as depicted in FIGS. 6A-6B, in accordance with one or more aspects of present disclosure. The method includes gaining access to the vasculature using an introducer sheath, such as the introducer sheath assembly described herein 1700. The vasculature can be accessed using known methods including using guidewires, dilators, and/or obturators to place the introducer sheath within the vasculature. As illustrated, the method also includes inserting the cannula sheath assembly into the introducer sheath assembly, such as through a lumen of the introducer sheath 1702. In one or more embodiments, a multi-durometer sealing gland, such as second multi-durometer sealing gland 310′, can be used to seal about the cannula sheath assembly within the introducer sheath connection hub 1704. In this embodiment, the hemostasis valve of the cannula sheath assembly, such as first multi-durometer scaling gland 310 of FIG. 6C is completely closed 1706. An aspiration source or device is coupled to the cannula sheath assembly 1708. In one or more embodiments, the aspiration source can be coupled via large-bore side port 600 of the cannula sheath connection hub 120′ (FIG. 6C). As illustrated, the process includes actuating the aspiration source or device coupled to the cannula sheath assembly to aspirate at least a portion of the undesired material via the cannula sheath assembly 1710. Once the undesired material is removed via the cannula sheath, it can pass through a filter, such as a filter described in the above-incorporated U.S. Patent Publication 2024/0148956. The filter removes the aspirated undesired material from the aspirated blood 1712. The filtered blood can be collected in a reservoir, and a syringe can be used to transfer the filtered blood from the reservoir to a side port of the introducer sheath so that the filter blood can be transferred from the syringe and reinfused into the patient via, for instance, side holes in the distal end of the introducer sheath 1714, such as described herein. In one or more embodiments, the reservoir can include a syringe, such that a separate reservoir to collect the filtered blood before being transferred to a syringe is not required.

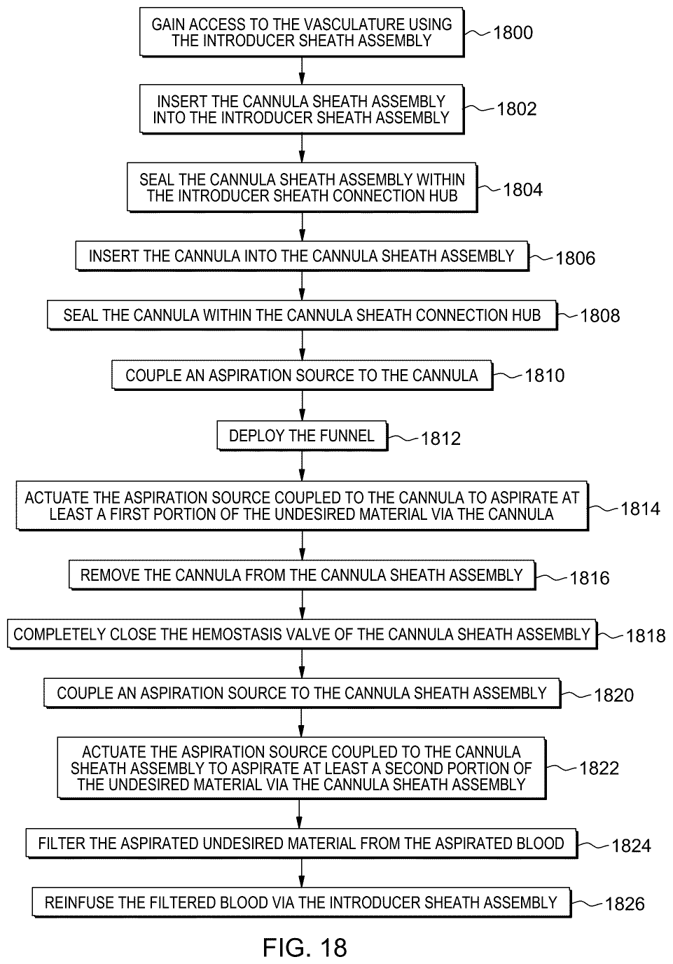

By way of further example, FIG. 18 depicts another embodiment of a method of removing an undesired material from a treatment site in combination with reinfusing of blood through an introducer sheath of an assembled aspiration system, such as depicted in FIGS. 8A-8B, in accordance with one or more aspects of present disclosure. The method includes accessing the vasculature using an introducer sheath 1800, such as the introducer sheath assembly described herein. The vasculature can be accessed using known methods including using guidewires, dilators, and/or obturators to place the introducer sheath within the vasculature. A cannula sheath assembly is inserted into the introducer sheath assembly, such as through a lumen of the introducer sheath 1802. In one or more embodiments, the process includes scaling about the cannula sheath assembly within the introducer sheath connection hub using, for instance, a multi-durometer scaling gland, such as second multi-durometer sealing gland 310′ (FIG. 4B) 1804. In one or more embodiments, the cannula is inserted into the cannula sheath assembly 1806, and another multi-durometer scaling gland, such as first multi-durometer scaling gland 310 (FIG. 4B), is used to seal about the cannula within the cannula sheath connection hub 1808. An aspiration source or aspiration device is coupled to the cannula 1810, and in one embodiment, the method also includes deploying a funnel at the distal end of the cannula 1812. In one or more embodiments, the cannula sheath can be pulled back to deploy the funnel at the distal end of the cannula, or the cannula can be pushed forward such that funnel is pushed out of the cannula sheath and deploys. The aspiration source(s) or device(s) is actuated to aspirate at least a first portion of the undesired material via the cannula 1814. The process further includes, in or more embodiments, removing the cannula from the cannula sheath assembly 1816, and completely closing the hemostasis valve, including the multi-durometer sealing gland, of the cannula sheath assembly 1818. In one or more embodiments, the method further includes coupling an aspiration source to the cannula sheath assembly 1820, such as to the large-bore side port of the cannula sheath assembly (FIGS. 8A-8B). The aspiration source or device coupled to the large-bore side port of the cannula sheath assembly is actuated to aspirate at least a second portion of the undesired material via the cannula sheath assembly 1822. The second portion of the undesired material may include a portion of the undesired material that remains within the vasculature after aspirating with the cannula, such as where the aspiration via the cannula did not remove a total amount of undesired material that is desired to be removed. In some embodiments, the second portion of the undesired material may include a portion of the undesired material that got lodged or stuck in the cannula sheath during the aspiration with the cannula. Examples of this aspect of the process are provided in FIGS. 10-13, and described further herein. As the undesired material is removed via the cannula and/or cannula sheath assembly, it can pass through a filter, such as a filter described in the above-incorporated U.S. Patent Publication 2024/0148956. In one or more embodiments, the filter captures the undesired material, while allowing filtered blood to pass through the filter 1824. The filtered blood can be collected in a reservoir and a syringe can be used to transfer the filtered blood. In one or more embodiments, the syringe can couple to the side port of the introducer sheath assembly such that the filtered blood can be transferred from the syringe and is reinfused into the patient via the side holes in the distal end of the introducer sheath 1826, such as described herein. In one or more embodiments, the reservoir can include a syringe, such that a separate reservoir to collect the filtered blood before being transferred to a syringe is not required.

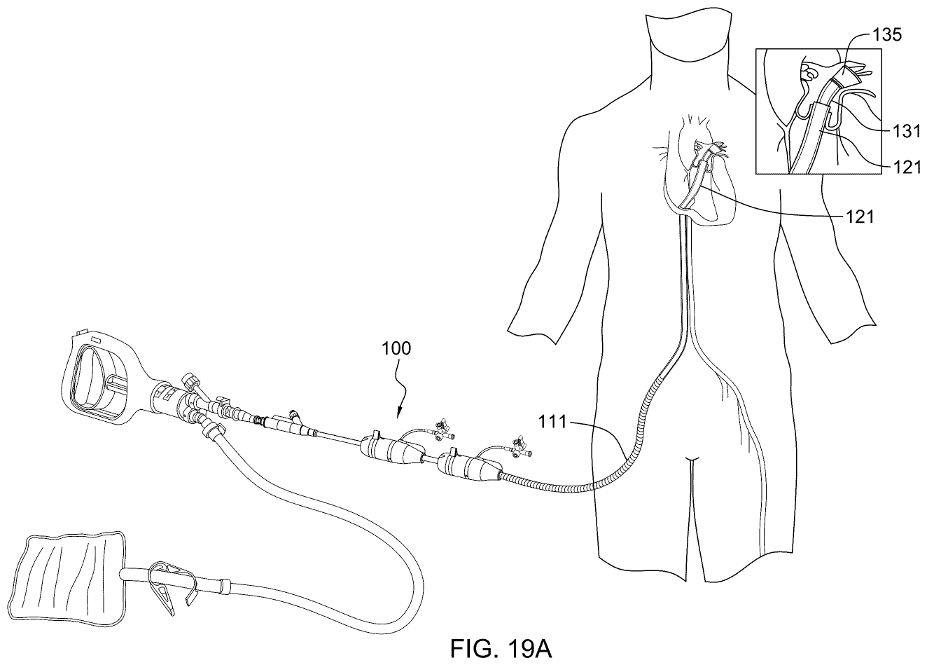

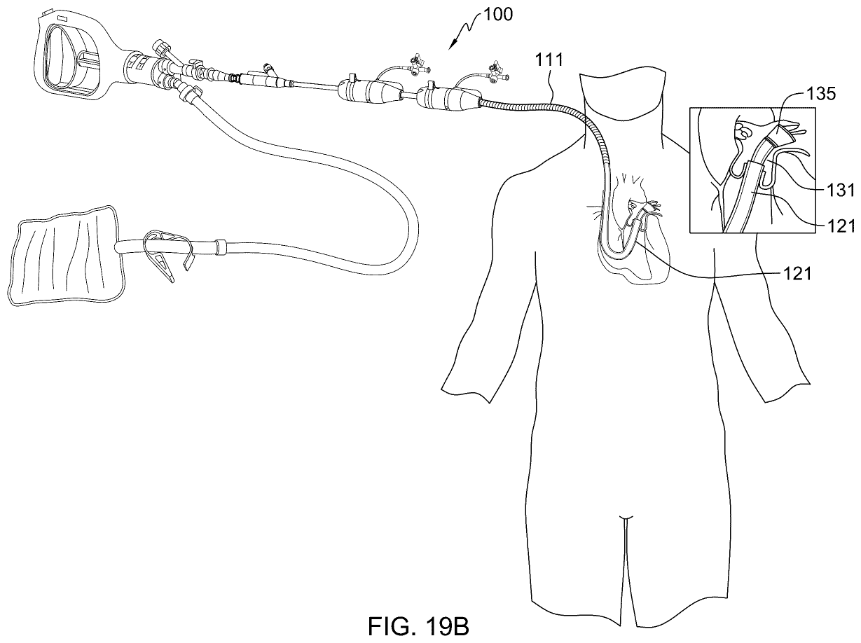

FIG. 19A shows the system 100 being used to treat a pulmonary embolism (PE) by accessing the vasculature through the leg (e.g., using a standard seldinger technique) to access the femoral artery. As shown, the distal end of the introducer sheath 111 can be positioned proximate the bifurcation of the external iliac vein. However, it is to be understood that the distal end of the introducer sheath can be positioned anywhere desired by the clinician without departing from aspects of the disclosure. As such, injection of fluid and/or filtered blood through the introducer sheath 111 results in the fluid and/or filtered blood to be injected proximate the bifurcation. This is beneficial because the vessel at the injection site is large enough to handle the volume of infusion and is close to the heart. As shown, the distal end of the cannula sheath 121 can be advanced through the pulmonary valve and into the main pulmonary artery before the pulmonary artery branches to the right and left pulmonary arteries (e.g., within the saddle of the pulmonary artery), and the cannula 131 can be advanced distally out of the cannula sheath 121 within the pulmonary artery such that the funnel 135 can be deployed within the main pulmonary artery (or saddle of the pulmonary artery) and to the left and right branches of the pulmonary artery to remove the PE. Positioning the cannula sheath 121 within the pulmonic artery allows the clinical to retract and/or withdraw the cannula 131 as may be needed or determined to be clinical necessary, and easily reintroduce the cannula 131 within the pulmonary artery to continue treatment. For example, it may be necessary to withdraw the cannula 131 from the cannula sheath 121 to flush or de-clog the cannula 131. It may be necessary to retract the cannula 131 without completely withdrawing the cannula 131 from the cannula sheath 121 to reposition the cannula 131 within the pulmonary artery or switch to the other branch. In any scenario, however, positioning the cannula sheath 121 within the pulmonary valve holds the place of the system 100 within the desired treatment location to treat a PE.

FIG. 19B shows the system 100 being used to treat PE by accessing the vasculature through the base of the neck (e.g., using a standard seldinger technique) to access the internal jugular. As shown, the distal end of the introducer sheath 111 may be positioned proximate the heart (e.g., SVC (superior vena cava)). However, it is to be understood that the distal end of the introducer sheath can be positioned anywhere desired by the clinician without departing from aspects of the disclosure. As such, injection of fluid and/or filtered blood through the introducer sheath 111 results in the fluid and/or filtered blood to be injected proximate the heart. This is beneficial because the vessel at the injection site is large enough to handle the volume of infusion. In this approach, the funnel 135, cannula 131 and cannula sheath 121 can be positioned similarly to that described relative to FIG. 19A.

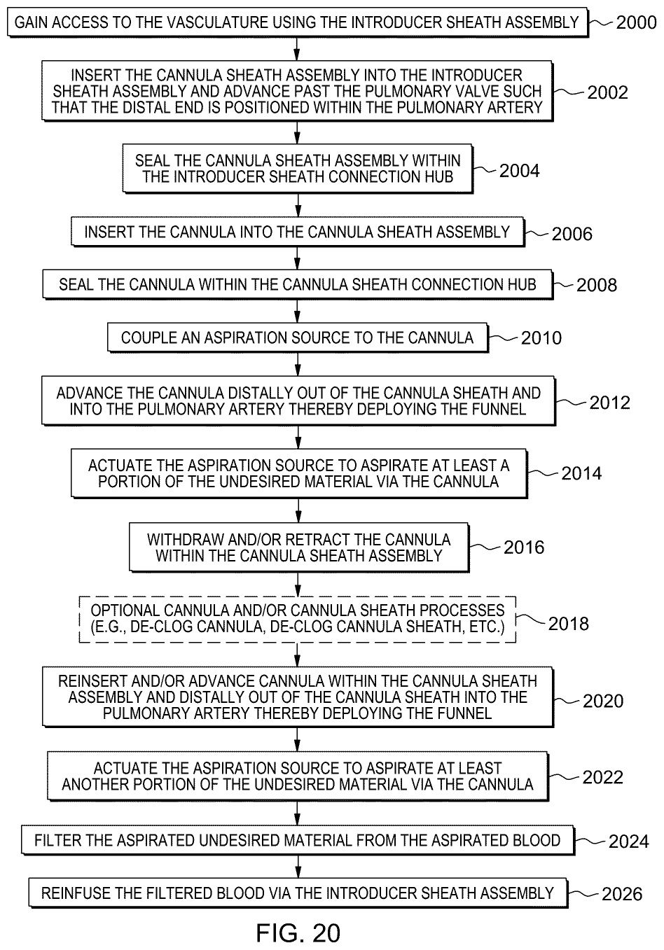

By way of further example, FIG. 20 depicts another embodiment of a method of removing an undesired material from a treatment site in combination with reinfusing of blood through an introducer sheath of an assembled aspiration system, such as depicted in FIGS. 8A-8B, and deployed, such as depicted in FIGS. 19A-19B, in accordance with one or more aspects of present disclosure. The method includes accessing the vasculature (e.g., the internal jugular or femoral artery) using an introducer sheath 2000, such as the introducer sheath assembly described herein. The vasculature can be accessed using known methods including using guidewires, dilators and/or obturators to place the introducer sheath within the vasculature. Referring to FIG. 19A, the vasculature can be accessed through the leg in some embodiments. Referring to FIG. 19B, the vasculature can be access through the base of the neck in other embodiments. A cannula sheath assembly is inserted into the introducer sheath assembly, such as through a lumen of the introducer sheath 2002. In one or more embodiments, the process includes sealing about the cannula sheath assembly within the introducer sheath connection hub using, for instance, a multi-durometer sealing gland, such as second multi-durometer sealing gland 310′ (FIG. 4B) 2004. In one or more embodiments, the cannula is inserted into the cannula sheath assembly 2006, and another multi-durometer sealing gland, such as first multi-durometer scaling gland 310 (FIG. 4B), is used to seal about the cannula within the cannula sheath connection hub 2008. An aspiration source or aspiration device is coupled to the cannula 2010. The method can also include, in one embodiment, advancing the cannula distally out of the cannula sheath and into the pulmonary artery to deploy a funnel at the distal end of the cannula 2012. In one or more embodiments, the cannula sheath can be pulled back to deploy the funnel at the distal end of the cannula, or the cannula can be pushed forward such that funnel is pushed out of the cannula sheath and deploys. The aspiration source(s) or device(s) is actuated to aspirate at least a first portion of the undesired material via the cannula 2014. The process further includes, in or more embodiments, withdrawing and/or retracting the cannula within and/or completely out of the cannula sheath assembly 2016. At this stage, the clinician may perform a number of optional clinical relevant processes 2018. In one example, the cannula can be de-clogged such as, for example, by actuating the aspiration source to aspirate any portion or debris of the undesired material remaining within the cannula or flushing the cannula. In some embodiments, this can also include advancing a secondary device such as an agitator or macerator into the cannula to disrupt clogged, undesired material therein. Alternatively or additionally, the cannula sheath can be de-clogged such as, for example, by operatively coupling an aspiration source to the large-bore side port of the cannula sheath and actuating the aspiration source to aspirate any portion or debris of the undesired material within the cannula sheath or flushing the cannula sheath. In some embodiments, this can also include advancing a secondary device such as an agitator or macerator into the cannula sheath to disrupt clogged, undesired material therein. The cannula can be reinserted and/or advanced within the cannula sheath assembly and distally out of the cannula sheath into the pulmonary artery thereby deploying the funnel 2020. The aspiration source can be actuated to aspirate at least another portion of the undesired material via the cannula 2022. As the undesired material is removed via the cannula and/or cannula sheath assembly, it can pass through a filter, such as a filter described in the above-incorporated U.S. Patent Publication 2024/0148956. In one or more embodiments, the filter captures the undesired material, while allowing filtered blood to pass through the filter 2024. The filtered blood can be collected in a reservoir and a syringe can be used to transfer the filtered blood. In one or more embodiments, the syringe can couple to the side port of the introducer sheath assembly such that the filtered blood can be transferred from the syringe and is reinfused into the patient via the side holes in the distal end of the introducer sheath 2026, such as described herein. Referring to FIG. 19A, the filtered blood can be reinfused proximate the bifurcation. Referring to FIG. 19B, the filtered blood can be reinfusion proximate the heart. In one or more embodiments, the reservoir can include a syringe, such that a separate reservoir to collect the filtered blood before being transferred to a syringe is not required.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprise” (and any form of comprise, such as “comprises” and “comprising”), “have” (and any form of have, such as “has” and “having”), “include” (and any form of include, such as “includes” and “including”), and “contain” (and any form contain, such as “contains” and “containing”) are open-ended linking verbs. As a result, a method or device that “comprises”, “has”, “includes” or “contains” one or more steps or elements possesses those one or more steps or elements, but is not limited to possessing only those one or more steps or elements. Likewise, a step of a method or an element of a device that “comprises”, “has”, “includes” or “contains” one or more features possesses those one or more features, but is not limited to possessing only those one or more features. Furthermore, a device or structure that is configured in a certain way is configured in at least that way, but may also be configured in ways that are not listed.

The corresponding structures, materials, acts, and equivalents of all means or step plus function elements in the claims below, if any, are intended to include any structure, material, or act for performing the function in combination with other claimed elements as specifically claimed. The description of one or more embodiments has been presented for purposes of illustration and description but is not intended to be exhaustive or limited to in the form disclosed. Many modifications and variations will be apparent to those of ordinary skill in the art. The embodiment was chosen and described in order to best explain various aspects and the practical application, and to enable others of ordinary skill in the art to understand various embodiments with various modifications as are suited to the particular use contemplated.

What is claimed is:

1. An aspiration system comprising:
a first sheath assembly comprising a first elongate shaft and a first hub at a proximal end of the first elongate shaft, wherein the first hub includes a hemostasis valve including a first multi-durometer sealing gland and a side port configured to be operatively coupled to a first suction source to generate a suction force through the first elongate shaft;
a cannula comprising a cannula shaft configured to be inserted within the first sheath assembly and to be operatively coupled to a second suction source to generate a suction force through the cannula, wherein the first multi-durometer sealing gland is configured to selectively close to seal around the cannula shaft or completely close when the cannula shaft is not positioned within the first sheath assembly, and
a second sheath assembly comprising a second elongate shaft having a tapered distal end and a second hub at a proximal end of the second elongate shaft, wherein the second hub includes a hemostasis valve having a second multi-durometer sealing gland, and wherein the first elongate shaft of the first sheath assembly is configured to be inserted into the second elongate shaft of the second sheath assembly and the second multi-durometer sealing gland of the second sheath assembly is configured to seal around the first elongate shaft of the first sheath assembly.

2. The aspiration system of claim 1, wherein the first sheath assembly is a cannula sheath assembly, and the second sheath assembly is an outer, introducer sheath assembly.

3. The aspiration system of claim 1, wherein at least one multi-durometer sealing gland of the first multi-durometer sealing gland and the second multi-durometer sealing gland comprises an intermediary layer disposed between outer layers, where the intermediary layer has a durometer different from a durometer of at least one outer layer of the outer layers.

4. The aspiration system of claim 3, wherein the durometer of the intermediary layer is lower than the durometer of the at least one outer layer.

5. The aspiration system of claim 1, wherein the first multi-durometer sealing gland comprises a first dual-durometer sealing gland, and the second multi-durometer sealing gland comprises a second dual-durometer sealing gland.

6. The aspiration system of claim 1, wherein the first suction source comprises one of a syringe or a manual mechanical aspiration source and the second suction source comprises one of the syringe or the manual mechanical aspiration source.

7. The aspiration system of claim 1, wherein the cannula shaft further comprises a funnel at a distal end of the cannula shaft to facilitate removal of an undesired material from a vessel of a subject.

8. The aspiration system of claim 1, wherein the cannula is configured to aspirate a first portion of an undesired material from a vessel of a subject via the cannula shaft, and wherein the first sheath assembly is configured to aspirate a second portion of the undesired material via the first elongate shaft and the side port of the first hub when the cannula shaft is not positioned within the first sheath assembly.

9. The aspiration system of claim 1, wherein the cannula further comprises a side access port at a proximal end to facilitate injection of a fluid or insertion of a device through the cannula into a vessel of a subject.

10. The aspiration system of claim 1, wherein the side port of the first hub of the first sheath assembly further includes an injection port to facilitate injection of a fluid or insertion of a device through the first elongate shaft into a vessel of a patient.

11. The aspiration system of claim 1, wherein at least one of the cannula shaft of the cannula or the first elongate shaft of the first sheath assembly comprises a collapsible lumen.

12. The aspiration system of claim 1, wherein the tapered distal end of the second elongate shaft of the second sheath assembly includes a plurality of openings to facilitate reinfusion of blood into a vasculature of a subject.

13. The aspiration system of claim 1, wherein the tapered distal end of the second elongate shaft is configured to seal around the first elongate shaft of the first sheath assembly when the first sheath assembly is operatively positioned within the second sheath assembly.

14. A method for removing an undesired material from a vasculature, the method comprising:
providing an aspiration system with a distal end proximate the undesired material, the aspiration system comprising:
a first sheath assembly comprising a first elongate shaft and a first hub at a proximal end of the first elongate shaft, wherein the first hub includes a hemostasis valve including a first multi-durometer sealing gland and a side port configured to be operatively coupled to a first suction source;
a cannula comprising a cannula shaft configured to be inserted within the first sheath assembly and to be operatively coupled to a second suction source, wherein the first multi-durometer sealing gland is configured to selectively close to seal around the cannula shaft or completely close when the cannula shaft is not positioned within the first sheath assembly, and
a second sheath assembly comprising a second elongate shaft having a tapered distal end and a second hub at a proximal end of the second elongate shaft, wherein the second hub includes a hemostasis valve having a second multi-durometer seal, and wherein the first elongate shaft of the first sheath assembly is configured to be inserted into the second elongate shaft of the second sheath assembly and the second multi-durometer seal of the second sheath assembly is configured to seal around the first elongate shaft of the first sheath assembly;
actuating the second suction source to generate a suction force through the cannula shaft to remove a first portion of the undesired material from a vessel of the vasculature;
removing the cannula shaft from the first sheath assembly; and
actuating the first suction source to remove a second portion of the undesired material from the vessel of the vasculature via the first elongate shaft and the side port of the first hub of the first sheath assembly.

15. The method of claim 14, wherein the first suction source comprises one of a syringe or a manual mechanical aspiration source.

16. The method of claim 14, wherein the tapered distal end of the second elongate shaft of the second sheath assembly includes a plurality of openings, and wherein the method further comprises reinfusing blood through the plurality of openings in the second elongate shaft of the second sheath assembly.

17. The method of claim 14, wherein the first multi-durometer sealing gland of the first hub seals against the cannula shaft with the second suction source actuated to remove the first portion of the material from the vessel through the cannula shaft, and is completely closed, or closed on a guidewire, with the cannula shaft removed from the first sheath assembly and the first suction source actuated to remove the second portion of the undesired material from the vessel via the first elongate shaft and the side port of the first hub of the first sheath assembly.

18. The method of claim 14, wherein the side port of the first hub of the first sheath assembly further includes an injection port, and wherein the method further comprises injecting a fluid or inserting a device through the injection port and first elongate shaft into the vessel.

19. The method of claim 14, wherein the cannula further comprises a side access port coupled to the cannula shaft, and the method further comprises injecting a fluid or inserting a device through the side access port and cannula shaft of the cannula into the vessel.

20. The method of claim 19, wherein the cannula shaft comprises a collapsible lumen, and wherein injecting the fluid inflates the collapsible lumen to allow the fluid to flow into the vessel.
